(19) United States
(12) Patent Application Publication
(10) Pub. No.: US 2024/0240208 A1
(43) Pub. Date: Jul. 18, 2024

(54) COMPOSITIONS AND METHODS FOR INCREASING EFFICIENCY OF PRECISE EDITING REPAIR

(71) Applicants: New York Genome Center, Inc., New York, NY (US); New York University, New York, NY (US)

(72) Inventors: Neville E. Sanjana, New York, NY (US); Zharko Daniloski, New York, NY (US)

(21) Appl. No.: 18/563,458

(22) PCT Filed: May 24, 2022
(86) PCT No.: PCT/US2022/072544

§ 371 Date: Nov. 22, 2023

Related U.S. Application Data
(60) Provisional application No. 63/192,277, filed on May 24, 2021.

(51) Int. Cl. C12N 15/90; C12N 9/22

(52) U.S. Cl. CPC C12N 15/907; C12N 9/22; C12N 2310/20

(57) ABSTRACT

Compositions and methods are provided for increasing the efficiency of precise gene editing of a target gene. One method includes administering to a mammalian subject in vivo or contacting mammalian cells ex vivo with a composition that temporarily inhibits, down-regulates, blocks or reduces the expression or activity of one or a combination of genes selected from Table 2 prior to or simultaneously with the components of a gene editing system. In one embodiment such components include a Cas enzyme and RNA guide for the precise editing repair of said target gene. In another embodiment such components include other DNA-targeting enzyme like TALE or ZFN for the precise editing repair of said target gene. Another method involves administering to a mammalian subject in vivo or contacting mammalian cells ex vivo with a composition that temporarily activates, up-regulates, stimulates or overexpresses the product, expression or activity of at least one or a combination of additional genes selected from Table 1 prior to or simultaneously with the components of a gene editing system for precise editing repair of said target gene, or any combination of inhibitors and activators. Still other methods include administering various combinations of such inhibiting and activating compositions.

### CROSS REFERENCE TO RELATED APPLICATIONS

This application claims the benefit under 35 USC § 119(e) of the priority of U.S. Patent Application No. 63/192,277, filed May 24, 2021. This application is hereby incorporated by reference in its entirety.

### STATEMENT REGARDING FEDERALLY SPONSORED RESEARCH OR DEVELOPMENT

This invention was made with government support under grant number D18AP00053 awarded by the Defense Advanced Research Projects Agency. This invention was made with government support under grant number DP2HG010099 awarded by the National Institutes of Health. The government has certain rights in this invention.

### BACKGROUND OF THE INVENTION

Gene editing therapies are a new class of gene therapies for precise repair of inborn genetic defects and disease prevention or reversal. A variety of gene editing systems are known including the zinc finger DNA-binding protein editing system or the Transcription

Activator-Like Effector-based Nuclease (TALEN) DNA-binding domain editing system as well as the Clustered regularly interspaced short palindromic repeats (CRISPR) genome editing system, and others. These techniques have been used to selectively activate/repress target genes, purify specific regions of DNA, image DNA in live cells, and precisely edit DNA and RNA. In brief, these editing systems binds a putative DNA or gene target.

Cleavage of the target results in a single-stranded break or a double-strand break (DSB) or nick in the gene target. The repair of the breaks and the editing of the specific target sequences depends on the type of repair strategy being used by a cell.

Nonhomologous DNA end joining (NHEJ) and homologous directed repair (HDR) are two major DNA repair pathways. The NHEJ repair pathway has been used to generate highly efficient insertions or deletions of variable-sized genes, but this repair system is error-prone and inaccurate. It frequently causes small nucleotide insertions or deletions (indels) at the DSB site that result in amino acid deletions, insertions, or frameshift mutations leading to premature stop codons within the open reading frame (ORF) of the targeted gene.

The HDR pathway uses homologous donor DNA sequences from sister chromatids or foreign DNA to create accurate insertions, base substitutions between double stranded breaks (DSB) sites created by the gene editing systems. This mechanism has high fidelity but low incidence. In order to utilize HDR for gene editing in the CRISPR techniques, for example, an exogenous DNA repair template containing the desired sequence to direct cleavage of the DNA must be delivered into the cell type of interest with the gRNA(s) and Cas9 or Cas9 nickase. Depending on the application and repair method, the repair template may be a single-stranded oligonucleotide, double-stranded oligonucleotide, or a double-stranded DNA plasmid. This can increase the probability of homologous recombination (HR) by about 1,000-fold. Notably, HDR can be used to accurately edit the genome in various techniques, including conditional gene knockout, gene knock-in, gene replacement, and point mutations. However, the efficiency of HDR is generally low (<10% of modified alleles). Other methods of precise gene repair include base editing or prime editing repair mechanisms.

A variety of methods have been reviewed for increasing the efficiency of precise gene repair. See, e.g., X-D. Tang et al., Methods for Activating Clustered Regularly Interspaced Short Palindromic Repeats/Cas9-Mediated Homology Directed Repair Efficiency, Frontiers in Genetics, 17 Jun. 2019, doi: 10.3389/fgene.2019.00551 and Liu, M., et al. (2019) Methodologies for Improving HDR Efficiency. Frontiers in genetics, 9, 691. Liu et al reviewed various methods of inhibiting NHEJ by using DNA ligase IV inhibitors or hindering certain gene expression with siRNA or shRNA, CRISPR-Cas delivery in the G2/S phase, adding homologous arms in donor templets and using modified Cas9. Also referenced were studies involving small molecules L755507, Brefeldin A, and RS-1, and over-expression of BRCA1 to increase HDR. Additionally, Cas9-CtIP, a fusion of Cas9 and CtIP, a protein involved in double-stranded break resection, can contribute to increased HDR efficiency.

Increasing precise editing repair efficiency in both ex vivo and in vivo environments will permit use of CRISPR or other gene editing systems in treating and correcting many DNA mutation-related diseases.

### SUMMARY OF THE INVENTION

Various compositions and methods are provided for improving efficiency of precise gene editing repair. In this specification for simplicity, we refer to the CRISPR gene editing system as an example of a gene editing technique or for gene editing components. It should be understood that wherever CRISPR is recited, another gene editing system and its components may also be used in place of CRISPR.

In one aspect, a composition comprises the components necessary for performing a genome editing technique and precise gene repair of a target gene, e.g., a target gene that is associated with a disease or disorder: and at least one inhibitory component that temporarily inhibits, down-regulates, or blocks the expression or activity of a gene selected from Table 2. In still other aspects, the composition includes at least one inhibitor of a gene involved in Non-homologous end-joining (NHEJ). In one aspect, the composition is designed for use in a Clustered regularly interspaced short palindromic repeats (CRISPR) gene editing system.

In another aspect, the composition comprises the components necessary for performing a Clustered regularly interspaced short palindromic repeats (CRISPR) genome editing technique and precise gene repair of a target gene that is associated with a disease or disorder: and at least one activating component that temporarily increases, upregulates or overexpresses the gene product or activity of a gene selected from Table 1.

In still another aspect, a composition comprises the components necessary for performing a Clustered regularly interspaced short palindromic repeats (CRISPR) genome editing technique and precise gene repair of a target gene that is associated with a disease or disorder: and a combination of at least one inhibitory component and at least one activating component identified herein. In still another aspect, the composition includes a combination with at least one inhibitor of a gene involved in Non-homologous end-joining (NHEJ).

The presence of the identified inhibitory and/or activating components, in various combinations in these compositions enables an increase in the efficiency of precise gene repair of the target gene.

In still another aspect, a method for increasing the efficiency of precise gene editing of a target gene comprises administering to a mammalian subject in vivo, or contacting mammalian cells ex vivo, with a composition that temporarily inhibits, down-regulates, blocks or reduces the expression or activity of one or a combination of genes selected from Table 2 prior to or simultaneously with components necessary to perform a CRISPR gene editing technique and CRISPR-mediated precise editing repair of said target gene. In still other aspects, the method includes at least one inhibitor of a gene involved in Non-homologous end-joining (NHEJ).

In still another aspect, a method for increasing the efficiency of precise gene editing of a target gene comprises administering to a mammalian subject in vivo, or contacting mammalian cells ex vivo, with a composition that temporarily activates, up-regulates, stimulates or overexpresses the product, expression or activity of at least one or a combination of additional genes selected from Table 1 prior to or simultaneously with the components necessary to perform a CRISPR gene editing technique and CRISPR-mediated precise editing repair of said target gene.

In another aspect, a method for increasing the efficiency of precise gene editing of a target gene comprises administering to a mammalian subject in vivo, or contacting mammalian cells ex vivo, a composition that includes both the inhibiting compositions or components described above and the activating compositions or components described herein prior to or simultaneously with the components necessary to perform a CRISPR gene editing technique and CRISPR-mediated precise editing repair of said target gene. In still other aspects, the method includes at least one inhibitor of a gene involved in Non-homologous end-joining (NHEJ).

Use of such compositions and methods for use in research and for the treatment of gene-associated disease is also an aspect of the inventions described herein.

Still other aspects and advantages of these compositions and methods are described further in the following detailed description of the preferred embodiments thereof.

### BRIEF DESCRIPTION OF THE DRAWINGS

FIG. 1 is a graph showing the results of validation of the top gene hits identified in the CRISPR inhibition (CRISPRi) screen of Example 1. Among the top CRISPRi hits that promote HDR we identified genes involved in DNA damage (DNA-PK, TP53BP1 and LIG4). Knock out of DNA-PK, TP53BP1 and LIG4 showed an increase in HDR levels, as previously established. We identified an additional 13 genes whose knock out promoted HDR in K562 cells. When NHEJ was blocked, the levels of HDR only increased to ˜50-60%.

FIG. 2A is a Western blot showing DNA-PK knock-out monoclonal cell lines in HEK293. DNA-PK monoclonal knockout cells were generated in HEK293 cells by targeting DNA-PK gene in HEK293 cells with a guide and Cas9 nuclease. Monoclonal lines were tested by western blot to check the expression of DNA-PK at protein level. Wildtype (WT) HEK293 cells show expression of DNA-PK, while the DNA-PK knockout was completely lost in clones 2, 3, 18, 19, and 22. Residual DNA-PK protein levels were detected in clone 1 and 24.

FIG. 2B is a bar graph showing the HDR levels in the DNA-PK monoclonal lines using the green fluorescent protein (GFP)-to-blue fluorescent protein (BFP) conversion assay. Levels of HDR were increased by 2-fold compared to WT cells and consistent across the monoclonal lines with complete loss of DNA-PK expression (clone 2, 3, 18, 19, 22).

FIG. 3 shows the results of a CRISPR inhibition screen. Using DNA-PK knockout clonal lines 18 and 22 as biological replicas, a genome-wide CRISPR inhibition screen was performed in these cells. The inventors identified genes that increase (rightmost third of FIG. 3) and decrease HDR (median fold change). Among the genes that decrease HDR (leftmost third of chart) are BRCA1, FANCM, FANCI, BARD1, and RBBP8.

FIG. 4A demonstrates that combinatorial gene perturbation drives significantly higher HDR levels. Knock out cell lines of the indicated genes were treated with 2 μM of DNA-PK inhibitor. HDR levels were determined by cell sorting. Blocking DNA-PK in RFC5, TUBA1B, NEDD8, LIG4, POLQ and RAD51 knock-out lines resulted in a significant increase in the HDR levels.

FIG. 4B is a bar graph showing additional results of arrayed validations using the GFP-to-BFP assay to determine increase in HDR resulting from inhibition of DNA-PK and a second gene target. Briefly, DNA-PK knockout cells clone 22 were targeted with NT (non-targeting guide as a control) to establish a baseline, or with a guide targeting one of the indicated genes. Most of the genes indicated under the X axis showed an increased HDR levels when perturbed in DNA-PK knockout cells. NT is the control: the targets are indicated. The red dotted line shows the results of inhibition of DNA-PK only on HDR. Precise repair levels are shown as a % of BFP+ cells over DMSO.

FIG. 5 is a list of gene targets selected from Table 2 and known small molecule inhibitors. The small molecule inhibitors were purchased from Selleckchem.com, Med ChemExpress and Millipore Sigma for these tests.

FIG. 6 is a graph of results of drug validations in DNA-PK KO cells clone 22 treated with dimethyl sulfoxide (DMSO) or 1 μM of the indicated inhibitors. Eighteen targets were targeted with 38 drugs. Some drugs were lethal and so were eliminated from use. Small molecule inhibitors were added in the media simultaneously with the introduction of Cas9, guide RNA and single-stranded DNA (ssDNA) encoding BFP. HDR levels were measured with the BFP-to-GFP assay. The drugs were washed off 24 hours later. ˜ 75% of the inhibitor compounds of the indicated gene targets showed an increased HDR levels. Precise repair levels are shown as a % of BFP+ cells over DMSO.

FIG. 7 is a heat plot showing dose dependent effects of small molecule inhibitors on HDR. DNA-PK KO cells were tested with the noted compounds at compounds at 10 μM, 5 μM, 1 μM, 0.5 μM, 0.1 μM, and 0.01 μM, as for FIGS. 4 and 6. HDR levels are shown as a % of BFP+ cells over DMSO 24 hours after drug treatment.

FIG. 8 is a heat plot showing cytotoxicity after drug treatment. HEK293 DNA-PK KO cells were treated with the noted compounds at 10 μM, 5 μM, 1 μM, 0.5 μM, 0.1 μM, and 0.01 μM in triplicate. After 24 hours an MTT assay was performed. Darker shading shows increasing percentage of viable cells as compared to DMSO.

FIG. 9A is a graph showing the BFP+ increase and cytotoxicity over DMSO for compound KPT-276. DNA-PK KO cells were tested with the noted compound at 10 μM, 5 μM, 1 μM, 0.5 M, 0.1 μM, and 0.01 μM, as for FIGS. 4 and 6. It was observed that inhibitors that promote HDR at low concentration are toxic at high concentration.

FIG. 9B is a graph showing the BFP+ increase and cytotoxicity over DMSO for compound SBE 13 HCl. DNA-PK KO cells were tested with the noted compound at 10 μM, 5 μM, 1 μM, 0.5 μM, 0.1 μM, and 0.01 μM, as for FIGS. 4 and 6. It was observed that for compounds that showed a dose-dependent increase in HDR, low toxicity was observed.

FIG. 10 shows a table of compound combinations. 11 compounds were selected from the results of the experiments described for FIGS. 7 and 8. These compounds are tested in combination at the noted concentrations.

### DETAILED DESCRIPTION

Methods and compositions are provided to enhance the efficiency of various techniques of precise gene repair. These methods and compositions involve the identification and combination of certain genes which when inhibited or activated, can increase the efficiency of one of more of the precise gene repair mechanisms. In certain embodiments, these compositions are used in combination with gene editing techniques, e.g., CRISPR, in a therapeutic setting. It is expected that such techniques are also useful in many clinical and research settings for increasing the efficiency of gene editing repair.

As described in the description and Examples and Figures herein, the inventors have identified certain human genes, which when the activity or expression of the gene product is inhibited or activated (i.e., over-expressed) can enhance forms of precise gene repair. In one embodiment, the form of precise gene repair that is enhanced in efficiency by these methods and compositions is homology-directed repair (HDR). In another embodiment, the form of precise gene repair that is enhanced in efficiency by these methods and compositions is nonhomologous DNA end joining repair. Other forms of precise gene repair are anticipated to respond to the same methods and compositions, including base editing repair and prime editing repair, as well as other forms of gene editing repair

### A. DESCRIPTION OF TERMS AND COMPONENTS OF THE METHODS AND COMPOSITIONS

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

By “Gene Editing System” is meant a system or technology which edits a target gene so as to alter, modify or delete the function or expression thereof. A genome editing system comprises at least one endonuclease component enabling cleavage of a target gene and at least one gene-targeting element. Examples of genome-targeting element include a DNA-binding domain (e.g., zinc finger DNA-binding protein or Transcription Activator-Like Effector-based Nuclease (TALEN) DNA-binding domain), guide RNA elements (e.g., CRISPR guide RNA), and guide DNA elements (e.g., NgAgo guide DNA) as described in US Patent Publication Application 2020/361877, incorporated by reference herein. Still other gene editing systems known to the art are intended to be encompassed by this term. As noted above, the use of the CRISPR gene editing system is intended to be representative of all other gene editing systems and components.

“CRISPR” or Clustered regularly interspaced short palindromic repeats genome editing techniques are useful for many types of genetic research, as well as treatment of diseases or disease conditions caused by malfunctioning or dysfunctioning genes. CRISPR is a gene editing system. In general, engineered CRISPR systems contain two components: a guide RNA (gRNA or sgRNA) and a CRISPR-associated endonuclease (Cas protein). The gRNA is a short synthetic RNA composed of a scaffold sequence necessary for Cas-binding and a user-defined ˜20 nucleotide spacer that defines the genomic target to be modified. When the gRNA and the Cas protein are expressed in the cell, the genomic target sequence to which they bind can be modified by an insertion or deletion or permanently disrupted. Additional information on CRISPR is provided in more detail in the Addgene CRISPR online guide (www.addgene.org/guides/crispr/) among multiple other known publications. See, also, U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830, US 2014-0287938 A1, US 2014-0273234 A1, US2014-0273232 A1, US 2014-0273231, US 2014-0256046 A1, US 2014-0248702 A1, US 2014-0242700 A1, US 2014-0242699 A1, US 2014-0242664 A1, US 2014-0234972 A1, US 2014-0227787 A1, US 2014-0189896 A1, US 2014-0186958, US 2014-0186919 A1, US 2014-0186843 A1, US 2014-0179770 A1 and US 2014-0179006 A1, US 2014-0170753: European Patents EP 2 784 162 B1 and EP 2 771 468 B1: European Patent Applications EP 2 771 468 (EP13818570.7), EP 2 764 103 (EP13824232.6), and EP 2 784 162 (EP14170383.5); and PCT Patent Publications PCT Patent Publications WO 2014/093661, WO 2014/093694, WO 2014/093595, WO 2014/093718, WO 2014/093709, WO 2014/093622, WO 2014/093635, WO 2014/093655, WO 2014/093712, WO2014/093701, WO2014/018423, WO 2014/204723, WO 2014/204724, WO 2014/204725, WO 2014/204726, WO 2014/204727, WO 2014/204728, WO 2014/204729, and WO2016/028682. These documents are all incorporated by reference to provide additional general information on CRISPR-Cas systems, components thereof, and delivery of such components, including methods, materials, delivery vehicles, vectors, particles, AAV, and making and using thereof, including as to amounts and formulations, some of which are useful in the present method and compositions or kits.

By the term “CRISPR components” as used herein is generally meant the gRNA and Cas protein. In one embodiment, the CRISPR components are selected from the type II CRISPR/Cas) genome editing system comprising Cas9 protein, CRISPR RNA (crRNA) and trans-activating crRNA (tracrRNA). A single-stranded guide RNA (sgRNA), a fusion of crRNA and tracrRNA, effectively recognizes specific sequences and directs the action of Cas) protein. The CRISPR components utilized in the compositions and methods described herein may also be selected from newer CRISPR/Cas systems that have been used for genome editing, including the type V Cas 12a system, and the endogenous type I and III CRISPR/Cas systems. These systems differ in protospacer adjacent motif (PAM) regions, Cas protein sizes, and cleavage sites. The type V CRISPR/Cas 12a genome editing system comprises crRNA and Cas 12a protein. Other Cas proteins are 12bk 12c and 14. Type I systems have the most cas genes, which are encoded by one or more operons. They contain six proteins, including the Cas3 protein which has helicase and nuclease activities. Multiple Cas proteins are combined with mature crRNA to form a CRISPR-associated complex for antiviral defense (Cascade), which binds to invading foreign DNA and promotes the pairing of crRNA and the complementary strand of exogenous DNA to form an R loop, which is recognized by Cas3 to cleave both the complementary and non-complementary strands. Type III systems contain the Cas10 protein with RNase activity and Cascade, and the function of Cascade resembles type I systems. Type III systems are categorized into four subtypes named A-D. Type IV Cas systems cleave RNA using Cas13. See, e.g., Liu, Z., et al. Application of different types of CRISPR/Cas-based systems in bacteria. Microb Cell Fact 19, 172 (2020); and Moon, S. B., et al. Recent advances in the CRISPR genome editing tool set. Exp Mol Med 51, 1-11 (2019), both incorporated by reference herein. Still other CRISPR components can include modified Cas proteins, such as Cas9 nickase, a D10A mutant of SpCas9, eSpCas9(1.1) and SpCas9-HF1, HypaCas9, evoCas9, xCas9 3.7 and Sniper-Cas (Addgene CRISPR Guide, cited above) or combinations thereof. It is anticipated that the compositions and methods of this invention can utilize CRISPR components and modified components of any suitable CRISPR/Cas system.

The term “Gene” is used in accordance with its customary meaning in the art. A gene is a sequence of nucleotides forming part of a chromosome, the order of which determines the order of monomers in a polypeptide or nucleic acid molecule which a cell (or virus) may synthesize. The term “Target Gene” as used herein refers to the gene which is targeted for gene editing. In certain embodiments, useful gene targets in the methods and compositions are those genes are involved in a genetically-mediated disease.

The term “Gene Product” refers to a sequence encoded by an identified gene having known function and/or activity. The Gene Product includes without limitation, fragments, isoforms, homologous proteins, oligopeptides, homodimers, heterodimers, protein variants, modified proteins, derivatives, analogs, and fusion proteins, among others. The proteins include natural or naturally occurring proteins, recombinant proteins, synthetic proteins, or a combination thereof with an identified function and/or activity. The term includes any recombinant or naturally occurring form of the Gene Product or variants thereof that maintain the known function or activity (e.g., within at least 30%, 40%, 50%, 60%, 70%, 80%, 90%, 95%, or 100% activity compared to wildtype protein). In embodiments, the gene product is a human gene product. See Table 1 and Table 2 for examples of genes and gene products useful in the compositions and methods described herein.

By the term “Precise Gene Repair” is meant any method that can be employed to repair the breaks in the nucleic acid target caused by the gene editing. As described above, the two primary repair pathways are NHEJ and HDR defined in the background. Other forms of repair include base editing and prime editing.

“Base Editing” uses components from CRISPR systems together with other enzymes to directly install point mutations into cellular DNA or RNA without making double-stranded DNA breaks (DSBs). This enables the efficient installation of point mutations in non-dividing cells without generating excess undesired editing byproducts. See, Rees H A, Liu D R. Base editing: precision chemistry on the genome and transcriptome of living cells. Nat Rev Genet. 2018 December; 19(12):770-788. Erratum in Nat Rev Genet. 2018 Oct. 19; PMID: 30323312; PMCID: PMC6535181. DNA base editors comprise a catalytically disabled nuclease fused to a nucleobase deaminase enzyme and, in some cases, a DNA glycosylase inhibitor. RNA base editors achieve analogous changes using components that target RNA.

“Prime Editing” is a targeted editing technique that facilitates insertions, deletions and conversions without breaking both strands of DNA and using DNA templates. See Anzalone A V et al. Search-and-replace genome editing without double-strand breaks or donor DNA. October 2019, Nature: 576; 149-157, incorporated by reference herein.

The term “Expression System” or “Delivery System” as used herein refers to the components and techniques for delivery the CRISPR components to, or expressing the CRISPR components in, a mammalian cell. These systems can include in vitro ex vivo or in vivo delivery. In one embodiment, a viral delivery system, which can also be used for in vivo delivery involves inserting the Cas protein and gRNA into a single lentiviral transfer vector or separate transfer vectors. Packaging and envelope plasmids provide the necessary components to make lentiviral particles. This well-known expression system can also provide stable tunable expression of the CRISPR components, including in vivo expression. In another frequently used viral expression system, the CRISPR components can be inserted in an AAV transfer vector and used to generate AAV particles. Other non-viral delivery systems include plasmid expression vectors using a Cas enzyme promoter that is constitutive (such as CMV, EF1alpha, CBh) or inducible (such as Tet-ON); or using a U6 promoter for gRNA can be used to transiently or stably express the Cas protein and/or gRNA in a mammalian cell. In yet another embodiment, RNA delivery of Cas protein and gRNA may be accomplished by in vitro transcription reactions to generate mature Cas mRNA and gRNA, which are then delivered to target cells through microinjection or electroporation. Yet another expression system is Cas9-gRNA ribonucleoprotein (RNP) complexes formed of purified Cas protein and in vitro transcribed gRNA combined into a complex. Such a complex can be delivered to cells using cationic lipids. In another embodiment, lipid nanoparticles (LNPs) are preferred, which predominantly target the liver. Messenger RNA (mRNA) encoding Cas9 and guide RNA, and a donor DNA template if necessary, is encapsulated into LNPs to shuttle these components to the liver.

“Lipid nanoparticle (LNPs)” generally refer to particles comprised of cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition. See, e.g., Fenton et al, Bioinspired Alkenyl Amino Alcohol Ionizable Lipid Materials for Highly Potent in vivo mRNA Delivery, Adv Mater. 2016 Apr. 20; 28(15): 2939-2943, which is incorporated herein by reference.

An “Activating Composition” as used herein refers to a mixture of at least one Activator of a gene or gene product of Table 1 with other chemical components, such as carriers, stabilizers, diluents, dispersing agents, suspending agents, thickening agents, and/or excipients suitable to the form of the activator, e.g., delivered in a plasmid or virus vs protein etc.

An “Inhibitory composition” as used herein refers to a mixture of at least one Inhibitor of a gene or gene product of Table 2 with other chemical components, such as carriers, stabilizers, diluents, dispersing agents, suspending agents, thickening agents, and/or excipients suitable to the form of the inhibitor, e.g., delivered as a siRNA vs protein etc.

A “Combined composition” also includes at least one Inhibitor of a gene or gene product of Table 2 and at least one Activator of a gene or gene product of Table 1 in one embodiment. Another embodiment includes at least one Inhibitor, at least one Activator and the CRISPR (or other gene editing) components. A composition facilitates administration of the Inhibitor and/or Activator/and/or CRISPR components to a cell in vitro, ex vivo or in vivo.

TABLE 1
CRISPRa

| Rank | Gene |
|---|---|
| 1 | USP17L19 |
| 2 | MLF1 |
| 3 | TRIB3 |
| 4 | MAGEA3 |
| 5 | GOLGA6D |
| 6 | SPRR2A |
| 7 | DENND5B |
| 8 | PDF |
| 9 | ZNF296 |
| 10 | TMEM136 |
| 11 | HIST1H2BM |
| 12 | KPNB1 |
| 13 | TMEM139 |
| 14 | SPI1 |
| 15 | IFNA16 |
| 16 | USP17L25 |
| 17 | MAP4K5 |
| 18 | KDELR1 |
| 19 | BBC3 |
| 20 | SH2D7 |
| 21 | SERPINB3 |
| 22 | MPHOSPH9 |
| 23 | SLC35G3 |
| 24 | GATA3 |
| 25 | CXorf38 |
| 26 | DNAH11 |
| 27 | CDV3 |
| 28 | RPL36AL |
| 29 | CXorf40B |
| 30 | OR2T35 |
| 31 | TGIF2LY |
| 32 | IFNA17 |
| 33 | DEFB107A |
| 34 | FOLH1 |
| 35 | PPM1A |
| 36 | YBEY |
| 37 | CXCL2 |
| 38 | ADH4 |
| 39 | LGALS7B |
| 40 | PRSS3 |
| 41 | ATXN7L3B |
| 42 | HIST1H2BL |
| 43 | PRB4 |
| 44 | VCY |
| 45 | KLK2 |
| 46 | IFT22 |
| 47 | LEUTX |
| 48 | RLN1 |
| 49 | WDHD1 |
| 50 | AMPD2 |
| 51 | OR10K1 |
| 52 | SH3BGRL |
| 53 | SNX24 |
| 54 | OTUD6A |
| 55 | SCO2 |
| 56 | HTN1 |
| 57 | OR2M7 |
| 58 | CSRP1 |
| 59 | CRIPAK |
| 60 | LRIG2 |
| 61 | MARVELD3 |
| 62 | TMEM265 |
| 63 | GABPA |
| 64 | RBMY1B |
| 65 | ZFP2 |
| 66 | UGT2B28 |
| 67 | ZNF592 |
| 68 | ZNF506 |
| 69 | RAP1GAP |
| 70 | TOM1 |
| 71 | RBMY1E |
| 72 | TNFRSF10B |
| 73 | FAM184A |
| 74 | RDH16 |
| 75 | SP110 |
| 76 | HAT1 |
| 77 | DEUP1 |
| 78 | OR51T1 |
| 79 | PLS1 |
| 80 | GSTA5 |
| 81 | ATP6V1H |
| 82 | MT1E |
| 83 | MTRNR2L2 |
| 84 | WDR41 |
| 85 | FOXO3 |
| 86 | FOXH1 |
| 87 | KRTAP9-7 |
| 88 | GIN1 |
| 89 | RBM19 |
| 90 | LOC105377134 |
| 91 | MBD3L4 |
| 92 | GABRA6 |
| 93 | NCAPG |
| 94 | ADGRE2 |
| 95 | TUBA3C |
| 96 | COX6B2 |
| 97 | PRAMEF4 |
| 98 | NOTCH2 |
| 99 | APOBEC3F |
| 100 | ZNF519 |
| 101 | GALNTL6 |
| 102 | KRTAP19-8 |
| 103 | PRR20B |
| 104 | OR2W3 |
| 105 | SULT6B1 |
| 106 | OR8B2 |
| 107 | MBD3L2 |
| 108 | OR10G4 |
| 109 | EIF3H |
| 110 | LOC441155 |
| 111 | ANKS1A |
| 112 | EPHB2 |
| 113 | PDIA3 |
| 114 | SLC10A4 |
| 115 | SLC50A1 |
| 116 | CAVIN3 |
| 117 | CPN1 |
| 118 | KANSL3 |
| 119 | NBPF3 |
| 120 | CDR2 |
| 121 | AGAP4 |
| 122 | ZFP64 |
| 123 | HNRNPCL4 |
| 124 | U2AF1L5 |
| 125 | NFX1 |
| 126 | CEBPD |
| 127 | ARL17A |
| 128 | TTC14 |
| 129 | DUSP7 |
| 130 | LMO1 |
| 131 | MUL1 |
| 132 | HLA-E |
| 133 | PRAMEF1 |
| 134 | COX15 |
| 135 | CLGN |
| 136 | RNF128 |
| 137 | CKAP5 |
| 138 | UBN1 |
| 139 | SUN3 |
| 140 | ENPEP |
| 141 | DSC2 |
| 142 | CXCR2 |
| 143 | POLR2B |
| 144 | P2RY13 |
| 145 | PSMA3 |
| 146 | PPP1R9B |
| 147 | PCDHA9 |
| 148 | COLEC11 |
| 149 | DDX60 |
| 150 | VRK2 |
| 151 | PARN |
| 152 | MAPK8IP3 |
| 153 | PPP1R12B |
| 154 | DPF1 |
| 155 | KRTAP10-10 |
| 156 | SNX1 |
| 157 | RFC3 |
| 158 | CYS1 |
| 159 | TMEM164 |
| 160 | NCAPH2 |
| 161 | APOL1 |
| 162 | JRK |
| 163 | SERPINE1 |
| 164 | RHOQ |
| 165 | TNFRSF10C |
| 166 | IMPG1 |
| 167 | NSG1 |
| 168 | ALG2 |
| 169 | RAB11A |
| 170 | OR4F17 |
| 171 | NBPF4 |
| 172 | SRPK1 |
| 173 | GLI2 |
| 174 | ERCC8 |
| 175 | SEC61A1 |
| 176 | OR2A42 |
| 177 | SHPK |
| 178 | YME1L1 |
| 179 | PNLDC1 |
| 180 | CLDN17 |
| 181 | NHLRC2 |
| 182 | RAD21 |
| 183 | KLC2 |
| 184 | PGM3 |
| 185 | DYRK1A |
| 186 | PIGH |
| 187 | VMP1 |
| 188 | WASHC2A |
| 189 | FRG2C |
| 190 | OR2G6 |
| 191 | TRAF6 |
| 192 | RBM24 |
| 193 | ZFAND6 |
| 194 | IPP |
| 195 | HEBP2 |
| 196 | TMEM14C |
| 197 | STX11 |
| 198 | PAM16 |
| 199 | FLJ45513 |
| 200 | FMNL2 |
| 201 | TDP1 |
| 202 | BRINP1 |
| 203 | ZMYM3 |
| 204 | AMIGO3 |
| 205 | ACAT2 |
| 206 | CAMK2N1 |
| 207 | UGT1A4 |
| 208 | ONECUT1 |
| 209 | REEP3 |
| 210 | NR4A2 |
| 211 | NAA50 |
| 212 | RTL5 |
| 213 | JPT1 |
| 214 | PSMD10 |
| 215 | METTL12 |
| 216 | ANKS4B |
| 217 | TMEM99 |
| 218 | POP1 |
| 219 | FAM133B |
| 220 | MRPS36 |
| 221 | MBOAT2 |
| 222 | NMBR |
| 223 | IKBKAP |
| 224 | CEP63 |
| 225 | DTWD2 |
| 226 | EIF2A |
| 227 | RNF17 |
| 228 | NGB |
| 229 | HIST1H2AI |
| 230 | GRIN2B |
| 231 | PKP3 |
| 232 | SCGB1C2 |
| 233 | CYP2A7 |
| 234 | RCBTB1 |
| 235 | PAPSS1 |
| 236 | WDR78 |
| 237 | KRT13 |
| 238 | FAM171A1 |
| 239 | HIST2H2AC |
| 240 | TSC22D2 |
| 241 | AMY2B |
| 242 | MUC17 |
| 243 | PCYOX1 |
| 244 | RALGDS |
| 245 | PDAP1 |
| 246 | CRX |
| 247 | CAV1 |
| 248 | NDUFB2 |
| 249 | CD96 |
| 250 | TMEM200A |
| 251 | A3GALT2 |
| 252 | NBPF9 |
| 253 | PGD |
| 254 | NETO1 |
| 255 | DMBX1 |
| 256 | TTC3 |
| 257 | ROPN1 |
| 258 | TMEM63B |
| 259 | LMNA |
| 260 | TMIGD1 |
| 261 | OR2A7 |
| 262 | PDIK1L |
| 263 | GRM6 |
| 264 | KIR3DL1 |
| 265 | TGM4 |
| 266 | GPR37 |
| 267 | LACTBL1 |
| 268 | ZAN |
| 269 | CTDSPL2 |
| 270 | CTSH |
| 271 | IFI44L |
| 272 | TMEM127 |
| 273 | GAGE2B |
| 274 | GGT7 |
| 275 | UGT1A8 |
| 276 | SH2D3C |
| 277 | ALX4 |
| 278 | EXOSC2 |
| 279 | ARHGEF10L |
| 280 | THEM4 |
| 281 | PSRC1 |
| 282 | SNTN |
| 283 | PRR16 |
| 284 | LRFN2 |
| 285 | CERS1 |
| 286 | ASAP1 |
| 287 | ZNF484 |
| 288 | GRAMD3 |
| 289 | PRODH2 |
| 290 | PRF1 |
| 291 | PLK4 |
| 292 | OR5D18 |
| 293 | TMEM169 |
| 294 | RABL2A |
| 295 | MOV10L1 |
| 296 | PHACTR4 |
| 297 | FERMT2 |
| 298 | GABRR2 |
| 299 | ST14 |
| 300 | RS1 |
| 301 | PABPC1L2B |
| 302 | AP2M1 |
| 303 | CAPN2 |
| 304 | KCNK18 |
| 305 | TRIM43 |
| 306 | HIF1A |
| 307 | HSFX1 |
| 308 | ATP11A |
| 309 | TXNDC5 |
| 310 | C4orf51 |
| 311 | CD8A |
| 312 | PFN2 |
| 313 | ADGRF4 |
| 314 | LRR1 |
| 315 | GPAT2 |
| 316 | GDI2 |
| 317 | PGPEP1L |
| 318 | PRSS48 |
| 319 | ARHGAP45 |
| 320 | NSDHL |
| 321 | PTPN20 |
| 322 | ULBP2 |
| 323 | PSG3 |
| 324 | CLCN3 |
| 325 | ZNF382 |
| 326 | B3GLCT |
| 327 | KRT6C |
| 328 | AGAP5 |
| 329 | VAMP5 |
| 330 | XRCC1 |
| 331 | SPSB3 |
| 332 | LMF1 |
| 333 | DCUN1D2 |
| 334 | ALDH3A2 |
| 335 | SAMD1 |
| 336 | C19orf57 |
| 337 | RPL7L1 |
| 338 | UBXN11 |
| 339 | UXS1 |
| 340 | CALB1 |
| 341 | URAD |
| 342 | NMRK1 |
| 343 | DNTTIP1 |
| 344 | STX7 |
| 345 | MCAT |
| 346 | HIST1H2BF |
| 347 | ZER1 |
| 348 | RAB12 |
| 349 | NOLC1 |
| 350 | SYNDIG1L |
| 351 | GRHL3 |
| 352 | MLNR |
| 353 | OR8B3 |
| 354 | ST18 |
| 355 | DDB2 |
| 356 | C1orf35 |
| 357 | ZNF395 |
| 358 | STEAP1 |
| 359 | EIF2B1 |
| 360 | LARP6 |
| 361 | MAPK1 |
| 362 | TAAR9 |
| 363 | ZNF728 |
| 364 | ADCK1 |
| 365 | YEATS2 |
| 366 | SWSAP1 |
| 367 | LANCL1 |
| 368 | LRRC40 |
| 369 | CYB5B |
| 370 | NCR1 |
| 371 | OR1S1 |
| 372 | CORIN |
| 373 | S1PR1 |
| 374 | MRPS27 |
| 375 | SLC27A6 |
| 376 | DUSP23 |
| 377 | TTPA |
| 378 | PNKP |
| 379 | ZNF479 |
| 380 | TSC22D4 |
| 381 | DEFB104B |
| 382 | ZNF552 |
| 383 | CTAGE4 |
| 384 | WBP2 |
| 385 | AGR3 |
| 386 | MOSPD3 |
| 387 | GTF3A |
| 388 | FGF14 |
| 389 | PTGER1 |
| 390 | PLEKHF2 |
| 391 | SPZ1 |
| 392 | RAC3 |
| 393 | ASIC5 |
| 394 | MTCH1 |
| 395 | VNN2 |
| 396 | PCDHB7 |
| 397 | KIAA1161 |
| 398 | GNAQ |
| 399 | P4HB |
| 400 | CYB5D1 |
| 401 | APBA2 |
| 402 | GCM1 |
| 403 | RPS10 |
| 404 | ADCK2 |
| 405 | SKA3 |
| 406 | TMEM92 |
| 407 | PNO1 |
| 408 | ATXN7L1 |
| 409 | VPREB3 |
| 410 | TNFRSF10A |
| 411 | UBAP2 |
| 412 | SLC24A1 |
| 413 | OSBPL9 |
| 414 | JSRP1 |
| 415 | RHCG |
| 416 | PDE10A |
| 417 | YIF1B |
| 418 | FAM161B |
| 419 | RFLNB |
| 420 | MSANTD2 |
| 421 | WDR45B |
| 422 | CEBPE |
| 423 | IL21 |
| 424 | SLC39A7 |
| 425 | MACF1 |
| 426 | KIF4A |
| 427 | STAB1 |
| 428 | CDCA4 |
| 429 | NACC2 |
| 430 | MBD4 |
| 431 | LTV1 |
| 432 | LOC157562 |
| 433 | ACE |
| 434 | PRAF2 |
| 435 | PTGDR2 |
| 436 | PIK3R1 |
| 437 | LRIT3 |
| 438 | ICOS |
| 439 | ZNF496 |
| 440 | PPP3R2 |
| 441 | DYNC2H1 |
| 442 | TTC33 |
| 443 | MAP9 |
| 444 | OR4A15 |
| 445 | NDUFB8 |
| 446 | ABCC10 |
| 447 | MTMR4 |
| 448 | ANK3 |
| 449 | TIMP2 |
| 450 | GATM |
| 451 | OR10V1 |
| 452 | PAQR4 |
| 453 | AEBP1 |
| 454 | OR5L2 |
| 455 | LPAR4 |
| 456 | FCMR |
| 457 | F8A1 |
| 458 | SPDYE3 |
| 459 | AEBP2 |
| 460 | MRC1 |
| 461 | MGMT |
| 462 | TEX264 |
| 463 | DUT |
| 464 | LOC101929726 |
| 465 | TCP10L2 |
| 466 | ASH1L |
| 467 | RPF1 |
| 468 | UBAP1 |
| 469 | CCDC38 |
| 470 | IL36RN |
| 471 | EDNRB |
| 472 | ANXA5 |
| 473 | FAM169A |
| 474 | ANXA6 |
| 475 | ANGEL2 |
| 476 | C1orf122 |
| 477 | NCOA3 |
| 478 | LTB4R |
| 479 | STYXL1 |
| 480 | GPR101 |
| 481 | EVL |
| 482 | C19orf47 |
| 483 | CYP2C19 |
| 484 | COL18A1 |
| 485 | ARL17B |
| 486 | BEND2 |
| 487 | PIMREG |
| 488 | CCDC103 |
| 489 | LPGAT1 |
| 490 | CGB8 |
| 491 | MS4A4E |
| 492 | C11orf91 |
| 493 | RPS14 |
| 494 | C1orf195 |
| 495 | NT5M |
| 496 | PREX1 |
| 497 | OR51A4 |
| 498 | RASSF1 |
| 499 | CFDP1 |
| 500 | MCMDC2 |

TABLE 2
CRISPRi

| Rank | Gene |
|---|---|
| 1 | PRKDC |
| 2 | RPL38 |
| 3 | SMU1 |
| 4 | HMGCR |
| 5 | MED12 |
| 6 | NEDD8 |
| 7 | RRP36 |
| 8 | RPL4 |
| 9 | SRP14 |
| 10 | BDP1 |
| 11 | RNGTT |
| 12 | PSMC2 |
| 13 | RPS4X |
| 14 | TPX2 |
| 15 | RPS10 |
| 16 | PES1 |
| 17 | BCS1L |
| 18 | GRWD1 |
| 19 | GTF2F2 |
| 20 | RFC5 |
| 21 | PSMA4 |
| 22 | MRPS26 |
| 23 | PMPCB |
| 24 | DAD1 |
| 25 | HAUS5 |
| 26 | MBD1 |
| 27 | PSMD3 |
| 28 | RACGAP1 |
| 29 | DDX20 |
| 30 | CWF19L2 |
| 31 | PNPT1 |
| 32 | NUTF2 |
| 33 | GRIN3B |
| 34 | MYC |
| 35 | FAM159A |
| 36 | CA1 |
| 37 | WDR61 |
| 38 | ACSBG1 |
| 39 | INTS3 |
| 40 | MRPL39 |
| 41 | EXOSC4 |
| 42 | PPP1R14A |
| 43 | NGDN |
| 44 | LRPPRC |
| 45 | DYNC1H1 |
| 46 | STK11IP |
| 47 | TUBA1B |
| 48 | GPR160 |
| 49 | FUT11 |
| 50 | EVC |
| 51 | RPA3 |
| 52 | LIAS |
| 53 | SLC35A3 |
| 54 | PFDN6 |
| 55 | OPRM1 |
| 56 | PI4KA |
| 57 | PCDH11X |
| 58 | CSTF3 |
| 59 | E4F1 |
| 60 | EFTUD2 |
| 61 | CCL13 |
| 62 | HMGCS1 |
| 63 | PROB1 |
| 64 | ASB6 |
| 65 | HLA-DOA |
| 66 | SFXN4 |
| 67 | WRN |
| 68 | LENG1 |
| 69 | NCBP2 |
| 70 | EDEM3 |
| 71 | CCR6 |
| 72 | C15orf41 |
| 73 | ZBTB2 |
| 74 | LOXL4 |
| 75 | RRM1 |
| 76 | ARL5C |
| 77 | RPS6 |
| 78 | TMEM9B |
| 79 | MOV10L1 |
| 80 | MTHFD1 |
| 81 | RPL10L |
| 82 | TCP1 |
| 83 | TCP11L1 |
| 84 | AURKA |
| 85 | ALG14 |
| 86 | IMP4 |
| 87 | RPL27 |
| 88 | CENPH |
| 89 | BRIX1 |
| 90 | KCMF1 |
| 91 | CFLAR |
| 92 | ABCA8 |
| 93 | ADGRB1 |
| 94 | GPATCH8 |
| 95 | RBBP5 |
| 96 | EP400 |
| 97 | PNISR |
| 98 | ZAR1 |
| 99 | MBP |
| 100 | TP53BP1 |
| 101 | TMEM65 |
| 102 | RNF40 |
| 103 | HSPA9 |
| 104 | GNG4 |
| 105 | DENND2A |
| 106 | OIT3 |
| 107 | EBI3 |
| 108 | OR52N2 |
| 109 | ZNF865 |
| 110 | ARHGEF17 |
| 111 | PCDH11Y |
| 112 | TUFM |
| 113 | PDE12 |
| 114 | EIF2S1 |
| 115 | METTL2A |
| 116 | TIMM44 |
| 117 | ZNHIT1 |
| 118 | AFG3L2 |
| 119 | FIGNL2 |
| 120 | PPCS |
| 121 | MED10 |
| 122 | BMS1 |
| 123 | CREG1 |
| 124 | COX8C |
| 125 | PKD2L2 |
| 126 | OLIG3 |
| 127 | BBS2 |
| 128 | FAM9A |
| 129 | BLOC1S5 |
| 130 | RANBP1 |
| 131 | SLC39A7 |
| 132 | GTF3C3 |
| 133 | PRCC |
| 134 | INCENP |
| 135 | INTS13 |
| 136 | WDR74 |
| 137 | WDR3 |
| 138 | C14orf119 |
| 139 | FEM1B |
| 140 | TUBGCP5 |
| 141 | CRYGD |
| 142 | JAK2 |
| 143 | RPH3AL |
| 144 | MAX |
| 145 | ARL1 |
| 146 | TATDN2 |
| 147 | FOXJ2 |
| 148 | NBR1 |
| 149 | WDR33 |
| 150 | RPL37A |
| 151 | SRPK1 |
| 152 | CPT1B |
| 153 | KRTAP2-4 |
| 154 | MED28 |
| 155 | PIGM |
| 156 | MYB |
| 157 | SUPT6H |
| 158 | SKA2 |
| 159 | ESCO2 |
| 160 | GP1BA |
| 161 | LDLRAP1 |
| 162 | CEP250 |
| 163 | DKC1 |
| 164 | FPGS |
| 165 | NBPF7 |
| 166 | CDH15 |
| 167 | MINDY2 |
| 168 | MRPL55 |
| 169 | COX11 |
| 170 | POLR1C |
| 171 | BRF1 |
| 172 | CPSF2 |
| 173 | APCDD1 |
| 174 | ZMAT2 |
| 175 | KCNK5 |
| 176 | TTC38 |
| 177 | PLS3 |
| 178 | ESPL1 |
| 179 | SMAD6 |
| 180 | GNB4 |
| 181 | ALMS1 |
| 182 | GTF3C1 |
| 183 | HMGB1 |
| 184 | PRPF19 |
| 185 | CCT6A |
| 186 | CNNM3 |
| 187 | SMN1 |
| 188 | WASHC2C |
| 189 | PKDREJ |
| 190 | RTF1 |
| 191 | GEMIN5 |
| 192 | DDX27 |
| 193 | OGFOD1 |
| 194 | NUP107 |
| 195 | TAF5 |
| 196 | CCDC174 |
| 197 | SDE2 |
| 198 | MYADM |
| 199 | PSMB6 |
| 200 | RPL3 |
| 201 | CYBRD1 |
| 202 | FLT1 |
| 203 | TMEM64 |
| 204 | MRPS25 |
| 205 | SRSF1 |
| 206 | RPL39 |
| 207 | PYROXD1 |
| 208 | AGAP2 |
| 209 | LOC100289561 |
| 210 | ZBED6CL |
| 211 | CFDP1 |
| 212 | HERC3 |
| 213 | TMCO3 |
| 214 | PTPRU |
| 215 | POP5 |
| 216 | C4orf51 |
| 217 | DERL1 |
| 218 | CHRND |
| 219 | HELZ |
| 220 | IP6K1 |
| 221 | SH3BP5 |
| 222 | TGIF2 |
| 223 | FTH1P18 |
| 224 | NDC80 |
| 225 | POLR2L |
| 226 | OR52B6 |
| 227 | TAF15 |
| 228 | NUFIP1 |
| 229 | MAPK7 |
| 230 | CKAP5 |
| 231 | PWP1 |
| 232 | OR51L1 |
| 233 | CASKIN1 |
| 234 | TBX22 |
| 235 | POLR2C |
| 236 | SLC22A7 |
| 237 | KLHL3 |
| 238 | SYNGR3 |
| 239 | PRRX2 |
| 240 | B3GLCT |
| 241 | SNRPG |
| 242 | MMP15 |
| 243 | SACS |
| 244 | GATD1 |
| 245 | RHOB |
| 246 | PSMC4 |
| 247 | PDLIM7 |
| 248 | ATP6V1B2 |
| 249 | MAGEB1 |
| 250 | GPR61 |
| 251 | CDK7 |
| 252 | UNC5CL |
| 253 | ZNF385B |
| 254 | PTPN11 |
| 255 | PGAM2 |
| 256 | LDLRAD4 |
| 257 | NUP98 |
| 258 | PCDHA10 |
| 259 | C9orf152 |
| 260 | RPS2 |
| 261 | SNRPB |
| 262 | MRPL37 |
| 263 | UBA6 |
| 264 | PTCD3 |
| 265 | USP51 |
| 266 | OR8H1 |
| 267 | CPSF6 |
| 268 | IL10RB |
| 269 | RBM11 |
| 270 | CHFR |
| 271 | PRSS3 |
| 272 | SPDL1 |
| 273 | PHF10 |
| 274 | BIRC6 |
| 275 | EIF2S2 |
| 276 | MAGEA12 |
| 277 | WDCP |
| 278 | DERL2 |
| 279 | SELENOH |
| 280 | RAD51 |
| 281 | OR6N2 |
| 282 | PPP3R1 |
| 283 | ZNF71 |
| 284 | ZBTB6 |
| 285 | NDUFA2 |
| 286 | EIF2AK1 |
| 287 | CCDC154 |
| 288 | OR51A4 |
| 289 | PSMB2 |
| 290 | RPL34 |
| 291 | TONSL |
| 292 | C20orf141 |
| 293 | GUK1 |
| 294 | SERGEF |
| 295 | POLR2G |
| 296 | RBM23 |
| 297 | CAMTA2 |
| 298 | TRMT6 |
| 299 | KLHL4 |
| 300 | MIA |
| 301 | PLA2G2D |
| 302 | CPNE1 |
| 303 | FTSJ3 |
| 304 | TXN |
| 305 | PRDM8 |
| 306 | FAM162A |
| 307 | TSEN15 |
| 308 | C1orf162 |
| 309 | SUCO |
| 310 | PNKP |
| 311 | GCNT4 |
| 312 | MATN1 |
| 313 | ATP6AP2 |
| 314 | OMD |
| 315 | CD8B |
| 316 | SPRY4 |
| 317 | FAM20C |
| 318 | CNTNAP3B |
| 319 | NENF |
| 320 | C7orf66 |
| 321 | CNPY3 |
| 322 | PCDHA6 |
| 323 | NMD3 |
| 324 | ZC3H18 |
| 325 | CRYBG1 |
| 326 | AHCTF1 |
| 327 | BHLHB9 |
| 328 | OAF |
| 329 | ZBTB34 |
| 330 | CDH8 |
| 331 | RHEB |
| 332 | GBF1 |
| 333 | LONRF2 |
| 334 | CTAG1B |
| 335 | OCSTAMP |
| 336 | CCNJ |
| 337 | TRAM2 |
| 338 | CABP5 |
| 339 | MYBBP1A |
| 340 | C17orf74 |
| 341 | OR13C5 |
| 342 | MRPL19 |
| 343 | AMY2B |
| 344 | ATP5E |
| 345 | NECAB1 |
| 346 | ABCE1 |
| 347 | ANKRD34A |
| 348 | GATA1 |
| 349 | TRAF3IP3 |
| 350 | TMEM141 |
| 351 | GPR78 |
| 352 | ATF3 |
| 353 | PTPMT1 |
| 354 | DDX23 |
| 355 | CDK12 |
| 356 | PTDSS1 |
| 357 | KIAA0040 |
| 358 | METTL2B |
| 359 | ZFPM1 |
| 360 | TMIE |
| 361 | KANSL1 |
| 362 | SP3 |
| 363 | SMG5 |
| 364 | CHCHD2 |
| 365 | XRCC5 |
| 366 | SLC29A4 |
| 367 | LIPC |
| 368 | TNFSF11 |
| 369 | ANKRD11 |
| 370 | CD3EAP |
| 371 | SAMD3 |
| 372 | CCNYL1 |
| 373 | HAUS6 |
| 374 | FNIP2 |
| 375 | C6orf62 |
| 376 | TBC1D20 |
| 377 | CTRC |
| 378 | OR10G4 |
| 379 | ATCAY |
| 380 | C9orf131 |
| 381 | PIEZO1 |
| 382 | FAM149B1 |
| 383 | TWISTNB |
| 384 | ASPM |
| 385 | UBN1 |
| 386 | CORO6 |
| 387 | BMP3 |
| 388 | DEFA1B |
| 389 | MED4 |
| 390 | SNRPC |
| 391 | MARCH5 |
| 392 | KRTAP5-1 |
| 393 | CTSA |
| 394 | CMIP |
| 395 | GYPA |
| 396 | AP5B1 |
| 397 | DOT1L |
| 398 | GALNT16 |
| 399 | ATP6V1E1 |
| 400 | TRPA1 |
| 401 | SEC22A |
| 402 | PLD3 |
| 403 | DOCK2 |
| 404 | TMEM225B |
| 405 | WBP11 |
| 406 | CNIH2 |
| 407 | FDFT1 |
| 408 | OR4B1 |
| 409 | STAT5B |
| 410 | PRAMEF25 |
| 411 | TUBG1 |
| 412 | LSG1 |
| 413 | TTC1 |
| 414 | PFDN4 |
| 415 | LIMS3 |
| 416 | NT5DC4 |
| 417 | LHX5 |
| 418 | FAM156A |
| 419 | UTP11 |
| 420 | CCNF |
| 421 | C5orf67 |
| 422 | LDAH |
| 423 | FAM47A |
| 424 | GOLGA6L22 |
| 425 | NAIF1 |
| 426 | TOMM34 |
| 427 | ZNF569 |
| 428 | HIST1H1E |
| 429 | CD40 |
| 430 | MAPK11 |
| 431 | PIEZO2 |
| 432 | KIF21B |
| 433 | CLEC2D |
| 434 | SLC12A5 |
| 435 | RAB34 |
| 436 | RAX2 |
| 437 | CBWD5 |
| 438 | ZFYVE26 |
| 439 | QARS |
| 440 | FOLH1B |
| 441 | OR7G3 |
| 442 | GAGE2E |
| 443 | RNF223 |
| 444 | PCDHGB1 |
| 445 | ATP50 |
| 446 | CERKL |
| 447 | CHRNB2 |
| 448 | OR8H3 |
| 449 | WNT6 |
| 450 | ZRSR2 |
| 451 | SBNO1 |
| 452 | SRGAP1 |
| 453 | OR6T1 |
| 454 | OR8B4 |
| 455 | HYAL2 |
| 456 | PLA2G16 |
| 457 | CACTIN |
| 458 | EPHA1 |
| 459 | SLC16A1 |
| 460 | ACOT6 |
| 461 | CLTCL1 |
| 462 | TOP1MT |
| 463 | CENPM |
| 464 | REXO4 |
| 465 | NUP93 |
| 466 | R3HDM1 |
| 467 | PPIL6 |
| 468 | CFAP36 |
| 469 | AGPAT2 |
| 470 | KRTAP19-5 |
| 471 | ANKRD50 |
| 472 | PRKCH |
| 473 | VPS13B |
| 474 | C8orf44 |
| 475 | RYK |
| 476 | SART3 |
| 477 | ZCCHC9 |
| 478 | ZNF551 |
| 479 | BEND2 |
| 480 | ST13 |
| 481 | IMPG2 |
| 482 | RRP15 |
| 483 | FAM71C |
| 484 | R3HCC1 |
| 485 | CLEC4D |
| 486 | MPP2 |
| 487 | CDKN2D |
| 488 | CASP7 |
| 489 | ELP3 |
| 490 | RBM28 |
| 491 | IFT27 |
| 492 | TAF2 |
| 493 | ERP29 |
| 494 | TTLL5 |
| 495 | CTNNBL1 |
| 496 | IRF2BP2 |
| 497 | TPP2 |
| 498 | EIF1AD |
| 499 | UBIAD1 |
| 500 | RPAP1 |

The terms “administering” and “administration” refer to the process by which a therapeutically effective amount of a compound, agent or composition contemplated herein is delivered to a cell or subject for research or treatment purposes. Multiple techniques of administering a compound exist in the art including, but not limited to, intravenous, oral, aerosol, parenteral, ophthalmic, pulmonary and topical administration. Guidance for preparing pharmaceutical compositions may be found, for example, in Remington: The Science and Practice of Pharmacy, (20th ed.) ed. A. R. Gennaro A. R., 2000, Lippincott Williams & Wilkins. Compositions are administered in accordance with good medical practices taking into account the subject's clinical condition, the site and method of administration, dosage, patient age, sex, body weight, and other factors known to physicians.

The terms “Priming” or “Pre-treating” or any variant thereof as used herein means administering or delivering to a cell ex vivo or subject in vivo, an Inhibiting Composition, an Activating Composition or a Combined Composition prior to delivering to the cell or subject the gene editing components, e.g., CRISPR Cas protein and gRNA, or substantially simultaneously therewith. In one embodiment, the term means administering or delivering to a cell ex vivo or subject in vivo, an Inhibiting Composition, an Activating Composition or a Combined Composition at least 1 to 24 hours prior to delivering to the cell or subject the gene editing components, e.g., CRISPR Cas protein and gRNA.

“Decrease”, “reduce”, “inhibit”, “down-regulate” are all used herein generally to refer to a decrease by a statistically significant amount. The decrease can be, for example, a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference level), or any decrease between 10-100% as compared to a reference level. The decrease or inhibition may be a decrease in activity, interaction, expression, function, response, condition, disease, or other biological parameter. This can include but is not limited to the complete ablation of the activity, interaction, expression, function, response, condition or disease.

“Activate”, “stimulate”, “over-express” “up-regulate” are all used herein generally to refer to an increase by a statistically significant amount. The increase can be, for example, a increase by at least 10% as compared to a reference level, for example a increase by at least about 20%, or at least about 30%, or at least about 40%, or at least about 50%, or at least about 60%, or at least about 70%, or at least about 80%, or at least about 90% or up to and including a 100% increase (e.g. absent level or non-detectable level as compared to a reference level), or any increase between 10-100% as compared to a reference level. The increase or activation may be an increase in activity, interaction, expression, function, response, condition, disease, or other biological parameter.

An “effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that may vary depending on one or more of: the particular agent chosen, the dosing regimen to be followed, whether it is administered in combination with other compounds, timing of administration, the tissue to be imaged, and the physical delivery system in which it is carried. As used herein, the effective amount of a composition containing an Inhibitor, and/or an Activator and/or Combined composition, as disclosed herein, is that effective to increase the efficiency of a selected precise gene repair of a target gene. Such results include, without limitation, the treatment of a disease or condition disclosed herein as determined by any means suitable in the art. In one embodiment, the effective amount of each Inhibiting compound and/or Activating compound is at least 1, 2, 3, 4, 5, 6, 7, 8, 9 and up to 10 or more micromolar concentration of a small molecule inhibitor/activator. Still other amounts can be determined to be effective by a physician with regard to the physical characteristics of the patient.

“Pharmaceutically acceptable” refers to those compounds, agents, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable carrier” includes any of the standard pharmaceutical carriers, such as a phosphate buffered saline solution, water, emulsions such as an oil/water or water/oil emulsion, and various types of wetting agents. The term also includes any of the agents approved by a regulatory agency such as the FDA or listed in the US Pharmacopeia for use in animals, including humans.

The terms “subject”, “individual” or “patient” refer, interchangeably, to a warm-blooded animal such as a mammal. In particular, the term refers to a human. A subject, individual or patient may be afflicted with, or suspected of having, or being pre-disposed to a genetically-mediated disease as described herein. The term also includes animals bred for food, as pets, or for study including horses, cows, sheep, poultry, fish, pigs, cats, dogs, and zoo animals, goats, apes (e.g., gorilla or chimpanzee), and rodents such as rats and mice.

The term “genetically-mediated disease” as used herein refers to any disease having a genetic origin, for which the gene causing or contributing to the disease, may be repaired by gene editing techniques. Such diseases, disorders, or conditions may be associated with an insertion, change or deletion in the amino acid sequence of the wild-type protein. Among such diseases are included inherited and/or non-inherited genetic disorders, as well as diseases and conditions which may not manifest physical symptoms during infancy or childhood. For example, www.uniprot.org/uniprot provides a list of mutations associated with genetic diseases, e.g., cystic fibrosis [www.uniprot.org/uniprot/P13569; also OMIM: 219700], MPSIH [http://www.uniprot.org/uniprot/P35475; OMIM:607014]; hemophilia B [Factor IX, http://www.uniprot.org/uniprot/P00451]; hemophilia A [Factor VIII, http://www.uniprot.org/uniprot/P00451]. Still other diseases and associated mutations, insertions and/or deletions can be obtained from reference to this database. Still other diseases are cancers having a genetic origin or due to a mutation in a wild-type gene. Embodiments of various cancers include but are not limited to carcinomas, melanomas, lymphomas, sarcomas, blastomas, leukemias, myelomas, osteosarcomas and neural tumors. In certain embodiments, the cancer is breast, ovarian, pancreatic or prostate cancer. Other diseases which are targets of gene editing treatments include glycogen storage disease type Ia (GSD Ia), Duchenne muscular dystrophy (DMD), myotonic dystrophy type 1 (DM1). Other suitable diseases for treatment with gene editing and thus suitable for these methods and compositions are listed in, e.g., http://www.genome.gov/10001200; http://www.kumc.edu/gec/support/; http://www.ncbi.nlm.nih.gov/books/NBK22183/. Clinical trials are already in process using CRISPR to treat cancers having a genetic component, such as non-small cell lung cancer: blood disorders such as beta-thalassemia and sickle cell disease and hemophilia, hereditary causes of blindness such as Leber congenital amaurosis, AIDS, cystic fibrosis, muscular dystrophy, Huntington's disease and viral diseases. See, e.g., C. R. Fernandez, Eight Diseases CRISPR Technology Could Cure, Best in Biotech. Labiotech.eu (April 2021)

As used herein and in the appended claims, the singular forms “a”, “an”, and “the” include plural reference unless the context clearly dictates otherwise. As such, the terms “a” (or “an”), “one or more,” and “at least one” are used interchangeably herein.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The words “consist”, “consisting”, and its variants, are to be interpreted exclusively, rather than inclusively, i.e., to exclude components or steps not specifically recited.

As used herein, the term “about” means a variability of plus or minus 10% from the reference given, unless otherwise specified.

### B. METHOD OF PRETREATMENT PRIMING WITH INHIBITORY COMPOUND(S)

In one embodiment, a method for increasing the efficiency of precise gene editing of a target gene comprises priming or pre-treating a mammalian cell that is intended to be subjected to gene editing, by delivering to the cell an inhibitory composition or inhibitory component or compound that temporarily inhibits, down-regulates, blocks or reduces the expression or activity of a selected gene or gene product. In one embodiment, a combination of selected inhibitory compositions is delivered. In certain embodiments, each inhibitory component or compound in the composition inhibits one gene or gene product. Certain combinations of two or more genes or gene products may be inhibited by combinations of two or more inhibitory compositions.

In one embodiment, the priming or pre-treating step occurs simultaneously with the delivery of components necessary to perform a gene editing technique and precise editing repair of the target gene. In one embodiment, the priming or pre-treating step occurs simultaneously with the delivery of CRISPR components (e.g., Cas protein and gRNA) necessary to perform a CRISPR gene editing technique and precise editing repair of the target gene. In another embodiment, the priming or pre-treating step occurs prior to the delivery of components necessary to perform a gene editing technique and precise editing repair of the target gene. In one embodiment, the priming or pre-treating step occurs prior to delivery of the components necessary to perform a CRISPR gene editing technique and CRISPR-mediated precise editing repair of the target gene. In one embodiment, the priming or pre-treating step occurs prior 1 to 24 hours prior to delivery of the components necessary to perform a CRISPR gene editing technique and CRISPR-mediated precise editing repair of the target gene. In another embodiment, the inhibitory compounds are delivered in a single composition with the gene editing, e.g., CRISPR, components.

These methods for increasing the efficiency of precise gene editing of a target gene can include delivering to a mammalian cell in vitro or ex vivo the inhibitory composition(s) or inhibitory component(s) or compound(s) by delivering the CRISPR components to a cell for manipulation of the target gene outside of the body. These methods for increasing the efficiency of precise gene editing of a target gene can also include administering or delivering the components of the CRISPR system and the inhibitory composition(s) in vivo to a mammalian subject.

The inhibitory compositions describe herein temporarily inhibit, down-regulate, block or reduce the expression or activity of a gene or gene product. The gene(s) or gene product(s) are identified in rank order in the list of Table 2. In one embodiment, the gene(s) or gene product(s) are identified in rank order from the top 250 genes in the list of Table 2. In another embodiment, the gene(s) or gene product(s) are identified in rank order from the top 100 genes in the list of Table 2. In another embodiment, the gene(s) or gene product(s) are identified in rank order from the top 50 genes in the list of Table 2. In another embodiment, the gene(s) or gene product(s) are identified in rank order from the top 25 genes in the list of Table 2. In another embodiment, the gene(s) or gene product(s) are identified in rank order from the top 15 genes in the list of Table 2. In another embodiment, the gene(s) or gene product(s) are identified in rank order from the top 10 genes in the list of Table 2. In another embodiment, the gene(s) or gene product(s) are identified in rank order from the top 5 genes in the list of Table 2.

In one embodiment, the inhibitory composition comprises an inhibitor of a gene selected from among DNA-PK, LIG4, TP53BP1, NEDD8, TUBA1B, SRPK1, RFC5, POLQ, RPL4, RANBP1, CDK7, CDK12, PRCC, RAD51, RRS10), WRN, RPA3, NUP98, MBD1, PPARG, SMC5, ESCO2, TATDN2, FIGNL1, PDS5A, or DDX5. In another embodiment, the inhibitory composition comprises an inhibitor of a gene involved in Non-homologous end-joining (NHEJ). In certain embodiments, the gene involved in NHEJ is DNA-PK. LIG4 or TP53BP1. In yet another embodiment, the inhibitory composition comprises an inhibitor of a gene(s) involved in NHEJ and an inhibitor of one or more additional genes of Table 2, wherein the combination of the temporary inhibition of the NHEJ gene and the temporary inhibition of one or more said additional genes increase the efficiency of said repair. In certain embodiments the inhibitory composition comprises an inhibitor of a gene(s) involved in NHEJ and an additional gene selected from POLQ, XPO1, RPL26, ARCN1, CACTIN, RPS24, TMA16, TWISTNB, CDC40, PSMD2, SNRPG, SMU1, CDK7 or NEPRO. In certain embodiments the inhibitory composition comprises an inhibitor of a gene(s) involved in NHEJ and an additional gene selected from MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9, STX18, MRPS5, INTS4, NUP 107, C6orf52 or HNRNPH2. In still other embodiments the inhibitory composition comprises an inhibitor of DNA-PK and an additional gene selected from POLQ, XPO1, RPL26, ARCN1, CACTIN, RPS24, TMA16, TWISTNB, CDC40, PSMD2, SNRPG, SMU1, CDK7, NEPRO, MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9), STX18, MRPS5, INTS4, NUP107, C6orf52 or HNRNPH2.

In still other embodiments the inhibitory composition comprises an inhibitor of DNA-PK and an additional gene selected from PLK1, AURKA, XPO1, CDK7, PSMC2, FNTA, BRD9, or PTGDR. Inhibitory composition(s) in still other embodiments employ two, three, four five, or more inhibitors that inhibit expression of two, three, four, five or more of the genes and respective gene products identified herein.

In still other embodiments the inhibitory composition comprises an inhibitor of a gene selected from PLK1, AURKA, XPO1, CDK7, PSMC2, FNTA, BRD9), or PTGDR. Inhibitory composition(s) in still other embodiments employ two, three, four five, or more inhibitors that inhibit expression of two, three, four, five or more of the genes and respective gene products identified herein.

In one embodiment, the inhibitor(s) is a small chemical molecule inhibitor(s) of the gene(s) or gene product(s), such as those listed small molecules listed in FIG. 5. In another embodiment, the inhibitor(s) is an siRNA or shRNA that targets the gene(s). In another embodiment, the inhibitor(s) is an anti-sense oligonucleotide. In another embodiment, the inhibitor(s) is delivered with the RNA-targeting enzyme Cas13. In still another embodiment, the inhibitor(s) is delivered in concert with CRISPR inhibition with Cas9, by delivering dCas9-repressor (KRAB, MeCP2, etc.) fusion protein to suppress expression of the gene product or its activity.

In certain embodiments, the inhibitor is SBE13 HCl. In another embodiment, the inhibitor is alisertib. In another embodiment, the inhibitor is LY3295688. In another embodiment, the inhibitor is MK-8745. In another embodiment, the inhibitor is KPT-276. In another embodiment, the inhibitor is YKL-5-124. In another embodiment, the inhibitor is VR-23. In another embodiment, the inhibitor is MG-132. In another embodiment, the inhibitor is FTI 277 HCl. In another embodiment, the inhibitor is BI-7273. In another embodiment, the inhibitor is setipiprant (ACT-129968). The structures of some desirable inhibitors are found in Table 3 below.

TABLE 3

| Compound | Structure |
|---|---|
| SBE 13 HCl | 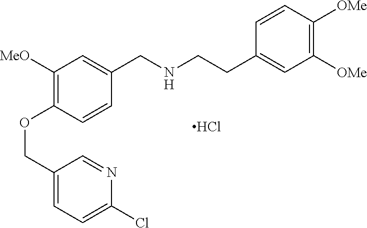 [SMILES: COc1ccc(CCNCc2ccc(OCc3ccc(Cl)nc3)c(OC)c2)cc1OC ; Cl] |
| Alisertib | 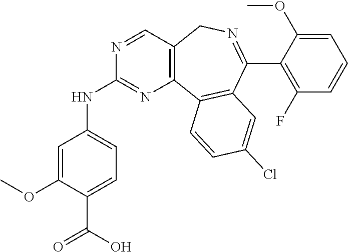 [SMILES: COc1cc(Nc2ncc3c(n2)-c2ccc(Cl)cc2C(c2c(F)cccc2OC)=NC3)ccc1C(=O)O] |
| LY3295668 | 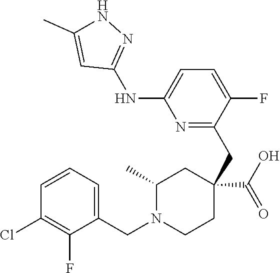 [SMILES: Cc1cc(Nc2ccc(F)c(C[C@@]3(C(=O)O)CCN(Cc4cccc(Cl)c4F)[C@H](C)C3)n2)n[nH]1] |
| MK-8745 | 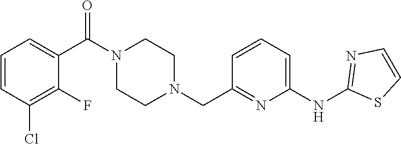 [SMILES: O=C(c1cccc(Cl)c1F)N1CCN(Cc2cccc(Nc3nccs3)n2)CC1] |
| KPT-276 | 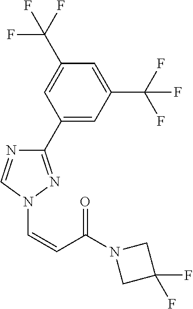 [SMILES: O=C(/C=C\n1cnc(-c2cc(C(F)(F)F)cc(C(F)(F)F)c2)n1)N1CC(F)(F)C1] |
| YKL-5-124 | 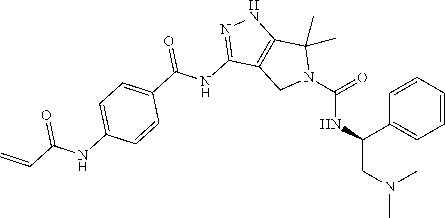 [SMILES: C=CC(=O)Nc1ccc(C(=O)Nc2n[nH]c3c2CN(C(=O)N[C@H](CN(C)C)c2ccccc2)C3(C)C)cc1] |
| VR23 | 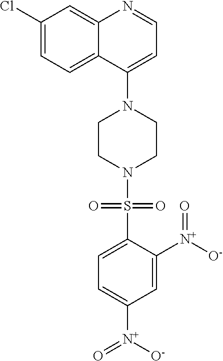 [SMILES: O=[N+]([O-])c1ccc(S(=O)(=O)N2CCN(c3ccnc4cc(Cl)ccc34)CC2)c([N+](=O)[O-])c1] |
| MG132 | 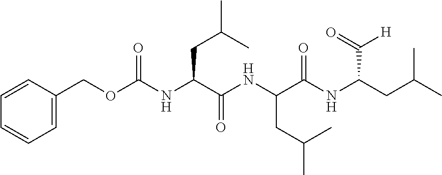 [SMILES: CC(C)CC(NC(=O)[C@H](CC(C)C)NC(=O)OCc1ccccc1)C(=O)N[C@H](C=O)CC(C)C] |
| FTI 277 HCl | 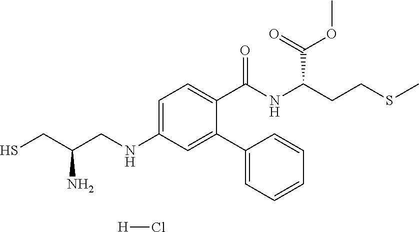 [SMILES: COC(=O)[C@H](CCSC)NC(=O)c1ccc(NC[C@@H](N)CS)cc1-c1ccccc1 ; Cl] |
| BI-7273 | 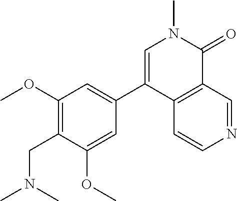 [SMILES: COc1cc(-c2cn(C)c(=O)c3cnccc23)cc(OC)c1CN(C)C] |
| Setipiprant | 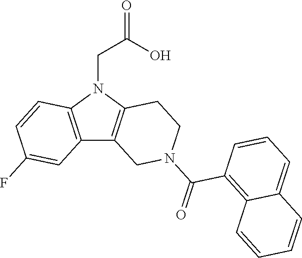 [SMILES: O=C(O)Cn1c2c(c3cc(F)ccc31)CN(C(=O)c1cccc3ccccc13)CC2] |

### C. METHOD OF PRETREATMENT PRIMING WITH ACTIVATING COMPOUND(S)

In one embodiment, a method for increasing the efficiency of precise gene editing of a target gene comprises priming or pre-treating a mammalian cell that is intended to be subjected to gene editing, by delivering to the cell an activating composition or activating component or compound that temporarily activates, up-regulates, stimulates or overexpresses the product, expression or activity of at least one additional gene or a combination of additional genes. In one embodiment, a combination of selected activating compositions is delivered. In certain embodiments, each activating component or compound in the composition activates, over-expresses or up-regulates one gene or gene product. Certain combinations of two or more genes or gene products may be activated, up-regulated, over-expressed or stimulated by combinations of two or more activating compositions.

In one embodiment, the priming or pre-treating step occurs simultaneously with the delivery of components necessary to perform a gene editing technique and precise editing repair of the target gene. In one embodiment, the priming or pre-treating step occurs simultaneously with the delivery of CRISPR components (e.g., Cas protein and gRNA) necessary to perform a CRISPR gene editing technique and precise editing repair of the target gene. In one embodiment, the priming or pre-treating step occurs prior to the delivery of components necessary to perform a gene editing technique and precise editing repair of the target gene. In another embodiment, the priming or pre-treating step occurs prior to delivery of the components necessary to perform a CRISPR gene editing technique and CRISPR-mediated precise editing repair of the target gene. In one embodiment, the priming or pre-treating step occurs prior 1 to 24 hours prior to delivery of the components necessary to perform a CRISPR gene editing technique and CRISPR-mediated precise editing repair of the target gene. In another embodiment, the activating compounds are delivered in a single composition with the gene editing, e.g., CRISPR, components.

These methods for increasing the efficiency of precise gene editing of a target gene can include delivering to a mammalian cell in vitro or ex vivo the activating composition or activating component or compound. These methods for increasing the efficiency of precise gene editing of a target gene can also include administering or delivering the components of the CRISPR system and the activating composition in vivo.

In another embodiment, a method for increasing the efficiency of precise gene editing of a target gene comprises administering to a mammalian cell an activating composition that temporarily inhibits, down-regulates, blocks or reduces the expression or activity of certain genes, gene products or combinations of such genes or gene products to a mammalian cell.

The activating compositions describe herein temporarily activate, stimulate, over-express or up-regulate the expression or activity of a gene or the amount of its gene product are used interchangeably throughout the specification. The gene(s) or gene product(s) are identified in rank order in the list of Table 1. In one embodiment, the gene(s) or gene product(s) are identified in rank order from the top 250 genes in the list of Table 1. In another embodiment, the gene(s) or gene product(s) are identified in rank order from the top 100 genes in the list of Table 1. In another embodiment, the gene(s) or gene product(s) are identified in rank order from the top 50 genes in the list of Table 1. In another embodiment, the gene(s) or gene product(s) are identified in rank order from the top 25 genes in the list of Table 1. In another embodiment, the gene(s) or gene product(s) are identified in rank order from the top 15 genes in the list of Table 1. In another embodiment, the gene(s) or gene product(s) are identified in rank order from the top 10 genes in the list of Table 1. In another embodiment, the gene(s) or gene product(s) are identified in rank order from the top 5 genes in the list of Table 1.

In one embodiment, the activating composition comprises two or more activating components, each component that temporarily increases, upregulates or overexpresses the gene product or activity of one gene selected from Table 1. In one embodiment the gene or gene product, which when over-expressed up-regulated, stimulated or activated causes an increase in precise gene repair is one of WDR77, RBBP8, RFC3, FANCB, BRCA1, RFC1, ATM, or FANCO2. Activating composition(s) in still other embodiments employ two, three, four five, or more activators that activate, over-express, up-regulate or stimulate expression of two, three, four, five or more of the genes and respective gene products identified herein.

In one embodiment, the activator(s) is a small chemical molecule inhibitor(s) of the gene(s) or gene product(s). Gene activation can be performed by delivering a fusion protein of the dCas9-activator (p65, HSF1, VP64 etc.) fusion protein, by delivering mRNA of the gene, by delivering the open reading frame (ORF) of the gene product expressed in a plasmid or recombinant virus as discussed herein or by delivery of the purified protein product of the gene. Other known methods of activating or overexpressing the indicated genes or gene activity are believed to be encompassed herein.

### D. METHOD OF PRETREATMENT PRIMING WITH COMBINATIONS OF INHIBITORY COMPOUND(S) AND ACTIVATING COMPOUND(S)

In another embodiment, a method for increasing the efficiency of precise gene editing of a target gene comprises priming or pre-treating a mammalian cell that is intended to be subjected to gene editing, by delivering to the cell an inhibitory composition or inhibitory component or compound that temporarily inhibits, down-regulates, blocks or reduces the expression or activity of a selected genes or gene product(s) and an activating composition or activating component or compound that temporarily activates, up-regulates, stimulates or overexpresses the product, expression or activity of at least one gene or a combination of additional genes or gene product(s). In one embodiment, one selected inhibitory composition is combined with one activating composition, each directed to a different gene or gene product. In another embodiment, the combination comprises two or more selected inhibitory compositions, each inhibiting, down-regulating, blocking or reducing the expression or activity of a combination of a selected gene or gene product and one activating composition.

In another embodiment, the combination comprises two or more selected activating compositions, directed toward activation of a different gene or gene product and one inhibiting composition. In still another embodiment, the combination comprises two or more selected inhibiting compositions with two or more selected activating compositions, with each composition directed toward inhibition or activation of a different gene or gene product. In certain embodiments, each inhibitory component or compound in the composition inhibits one gene. In certain embodiments, each activating component or compound in the composition activates one gene.

In one embodiment, the priming or pre-treating step occurs simultaneously with the delivery of components necessary to perform a gene editing technique and precise editing repair of the target gene. In one embodiment, the priming or pre-treating step occurs simultaneously with the delivery of CRISPR components (e.g., Cas protein and gRNA) necessary to perform a CRISPR gene editing technique and precise editing repair of the target gene. In one embodiment, the priming or pre-treating step occurs prior to the delivery of components necessary to perform a gene editing technique and precise editing repair of the target gene. In another embodiment, the priming or pre-treating step occurs prior to delivery of the components necessary to perform a CRISPR gene editing technique and CRISPR-mediated precise editing repair of the target gene. In one embodiment, the priming or pre-treating step occurs prior 1 to 24 hours prior to delivery of the components necessary to perform a CRISPR gene editing technique and CRISPR-mediated precise editing repair of the target gene. In one embodiment, the inhibitory components of the combined composition are delivered via a different delivery system than the activating components. In one embodiment the inhibitory components of the combined composition are delivered via the same form of delivery system. In another embodiment the inhibitory components of the combined composition are simultaneously or sequentially with the activating components. In another embodiment, the combination of inhibitory compound(s) and activating compound(s) are delivered in a single composition, prior to or simultaneously with the gene editing components. In another embodiment, the combination of inhibitory compound(s) and activating compound(s) are delivered in a single composition with the gene editing, e.g., CRISPR, components.

These methods for increasing the efficiency of precise gene editing of a target gene can include delivering to a mammalian cell in vitro or ex vivo the combination composition(s) by delivering the CRISPR components to a cell for manipulation of the target gene outside of the body. These methods for increasing the efficiency of precise gene editing of a target gene can also include administering or delivering the components of the CRISPR system and the combination inhibitor/activator composition(s) in vivo to a mammalian subject.

In one embodiment, the inhibitory components of the combined composition are selected from the lists of Table 2 as described herein. In one embodiment, the activating components of the combined composition are selected from the lists of Table 1 as defined above. In one embodiment, the combined composition comprises an inhibitor of one or more of the genes selected from among DNA-PKcs, LIG4, TP53BP1, NEDD8, TUBA1B, SRPK1, RFC5, POLQ, RPL4, RANBP1, CDK7, CDK12, PRCC, RAD51, RRS10, WRN, RPA3, NUP98, MBD1, PPARG, SMC5, ESCO2, TATDN2, FIGNL1, PDS5A, or DDX5 and an activator of one or more of the genes selected from WDR77, RBBP8, RFC3, FANCB, BRCA1, RFC1, ATM, or FANCO2. In another embodiment, the combined composition comprises an inhibitor of a gene involved in Non-homologous end-joining (NHEJ), an inhibitor of at least one other gene of Table 2, and an activator of at least one gene of Table 1. In certain embodiments, the gene involved in Non-homologous end-joining (NHEJ) is DNA-PK, LIG4 or TP53BP1. In another embodiment, the combined composition comprises an inhibitor of DNA-PK, an inhibitor of one or more of the genes selected from among, LIG4, TP53BP1, NEDD8, TUBA1B, SRPK1, RFC5, POLQ, RPL4, RANBP1, CDK7, CDK12, PRCC, RAD51, RRS10, WRN, RPA3, NUP98, MBD1, PPARG, SMC5, ESCO2, TATDN2, FIGNL1, PDS5A, or DDX5 and an activator of one or more of the genes selected from WDR77, RBBP8, RFC3, FANCB, BRCA1, RFC1, ATM, or FANCO2.

One of skill in the art, given this disclosure may readily select more specific combinations of inhibitors and activators, optionally with an inhibitor of DNA-PK, LIG4 or TP53BP1 from among the known inhibitors to practice the claimed invention.

### E. COMPOSITIONS AND KITS

In some embodiments, kits and compositions provided herein are used to treat a subject having a genetically-mediated disease by editing a target gene or to edit any target gene in any therapeutic or non-therapeutic context. A composition or a kit suitable for the treatment of subject with a genetically-mediated disease includes, in one embodiment, the components necessary for performing a Clustered regularly interspaced short palindromic repeats (CRISPR) genome editing technique and precise gene repair of a target gene that is associated with a disease or disorder. The composition includes the Cas endonuclease and at least one gRNA that are able to bind the selected target gene. In one embodiment, the composition or kit includes an inhibitory component that temporarily inhibits, down-regulates, or blocks the expression or activity of a gene selected from Table 2. In another embodiment, the composition or kit includes an activating component that temporarily increases, upregulates or overexpresses the gene product or activity of a gene selected from Table 1. In yet a further embodiment, the composition or kit includes a combination of at least one inhibitor component and at least one activating component as defined above. The presence of Inhibiting component(s), the Activating component(s) or the combination of one or more of the Inhibiting component and Activating component in the composition or kit enables an increase in the efficiency of said precise gene repair of the target gene. The form of precise gene repair is homology-directed repair (HDR), nonhomologous DNA end joining repair, base editing repair, or prime editing repair among other repair formats.

In one embodiment, such a composition or kit comprises two or more inhibitory components, each component temporarily inhibiting, down-regulating, or blocking the expression or activity of one gene selected from Table 2. In one embodiment, the inhibitory component inhibits one or more of the gene selected from DNA-PKcs, LIG4, TP53BP1, NEDD8, TUBA1B, SRPK1, RFC5, POLQ, RPL4, RANBP1, CDK7, CDK12, PRCC, RAD51, RRS10, WRN, RPA3, NUP98, MBD1, PPARG, SMC5, ESCO2, TATDN2, FIGNL1, PDS5A, or DDX5. In one embodiment, the inhibitory component is selected from POLQ. XPO1, RPL26, ARCN1, CACTIN, RPS24, TMA16, TWISTNB, CDC40, PSMD2, SNRPG, SMU1, CDK7 or NEPRO. In another component, the inhibitory compound inhibits a gene selected from MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9, STX18, MRPS5, INTS4, NUP107, C6orf52 or HNRNPH2. In another embodiment, the inhibitory components comprise an inhibitor of a gene involved in Non-homologous end-joining (NHEJ), e.g., DNA-PK, LIG4 or TP53BP1, and one or more of the other inhibitors of a gene selected from Table 2. In certain embodiments, the inhibitory components are one or more of the small molecules of FIG. 5. In other examples, the inhibitor of DNA-PK is a compound identified in International Patent Publication Nos. WO2014/159690 and US Patent Application Publication No. 2020/361877, incorporated by reference herein.

The combination of the temporary inhibition of the NHEJ gene and the temporary inhibition of one or more additional genes increase the efficiency of said repair.

In another embodiment, the composition or kit comprises two or more activating components, each component that temporarily increases, upregulates or overexpresses the gene product or activity of one gene selected from Table 1. In one embodiment, the genes of Table 1, which when over-expressed or activated causes an increase precise gene repair are selected from WDR77, RBBP8, RFC3, FANCB, BRCA1, RFC1, ATM, or FANCO2.

In yet another embodiment, the composition or kit comprises a combination of inhibitory and activating components defined herein. In one embodiment, one of the inhibitory components is an inhibitor of a gene involved in Non-homologous end-joining (NHEJ), and is combined with a component that initiates over-expression of the products of one or more additional genes of Table 1, wherein the combination of the temporary inhibition of the NHEJ gene and the temporary overexpression of the product of Table 1 gene increases the efficiency of the repair.

Still other embodiments contain an inhibitor of the NHEJ gene, e.g., DNA-PK, an inhibitor of another gene from Table 2 identified above, and at least one activator of a gene selected from Table 1 above.

The composition or kit also can contain a delivery vehicle suitable for administration in vivo into a mammalian subject. In another embodiment, the composition or kit also can contain a delivery vehicle suitable for administration ex vivo to cells of a mammalian subject. The expression form of the inhibitor or activator can be, independently, a small molecule, an mRNA or DNA encoding the additional gene, a plasmid, a recombinant virus, an siRNA, an shRNA, a second RNA guide sequence directed to an additional gene, a purified protein product, an antibody, a plasmid or recombinant virus expressing the component as a DNA or protein, or combinations thereof. Depending upon the nature of the inhibitory compound and/or activating compound, the delivery vehicle is a polymeric nanoparticle, inorganic nanoparticle, a lipid-based composition, a nanocapsule lipid base, a recombinant viral vector, a recombinant plasmid, a pharmaceutically acceptable buffer, or combinations thereof.

In one embodiment, the composition or kit contains the Cas enzyme and RNA guides for repair of the target gene packaged in a nanoparticle or nanocapsule and delivered to the subject or cells separately from the inhibitory and/or activating components.

### F THERAPEUTIC METHODS

The methods and compositions described above can be used to increase precise gene repair efficiency in a therapeutic setting to improve the treatment of genetically-mediated disease in a mammalian subject. In one embodiment, the subject is a human patient with a genetically-mediated disease.

In one embodiment, the methods and compositions may be used to pretreat a cell ex vivo. An autologous mammalian T cell, bone marrow cell or cell of any tissue is obtained from the mammalian subject and pre-treated with an effective amount of the appropriate Inhibiting. Activating or Combined composition described herein. Once the gene editing components, e.g., CRISPR components, are delivered to the cell ex vivo, the target gene in the cell is corrected by insertion, deletion or replacement. The treated cell is subsequently transferred in vivo to the mammalian subject. In one embodiment, the pre-treated/edited cell is delivered systemically to the subject. In another embodiment, the pre-treated/edited cell is delivered to a desired targeted tissue. This method can be applied to CAR T cells or cells of any tissue or organ having a target gene that requires editing to treat a disease.

In other methods, now in practice in clinical trials, the compositions may be administered in vivo to the subject using viral delivery methods, such as by AAV or lentivirus. See, e.g., US Patent Publication Application 2020/361877 and publications cited therein, incorporated by reference.

It is anticipated that other delivery methods, as developed, will be used to deliver the compositions and components of this invention, without under experimentation in view of the disclosure herein.

### G. EXAMPLES

The following examples disclose specific embodiments of inhibiting certain gene targets to increase efficiency of HDR in CRISPR gene editing settings. These examples encompass any and all variations that become evident as a result of the teaching provided herein.

### Example 1—Original Crispr Inhibition and Activation Screens

The inventors identified regulators of homologous directed repair (HDR) that synergize with inhibition of non-homologous end-joining (NHEJ) and further increase HDR levels in human cells. The inventors targeted all of the 20,000 genes in the human genome using a pair of CRISPR-based screens to identify genes that, upon loss (knock-out) or gain (overexpression), increase precise gene repair. Using a CRISPR inhibition screen, a ranked list of the effect of loss of every human gene on precise gene repair. In a CRISPR activation screen, we produced a ranked list of the effect of gain of every human gene on precise gene repair. Combinatorial effects of multiple gene/drug perturbations on boosting precise gene repair were also examined. A set of the cell lines carrying a specific gene knock-out were treated with 2 μM DNA-PK small molecule inhibitor. Inhibiting DNA-PK in RFC5, TUBA1B, NEDD8, LIG4, POLQ and RAD51 knock out cell lines resulted in a significant increase in the HDR levels. Combinatorial genes perturbation resulted in HDR levels as high as 75%, which is ˜3-fold increase compared to the control cells.

To identify the genes required for efficient homology-directed repair (HDR), we performed two genome-wide CRISPR screens: CRISPR inhibition screen (CRISPRi) using Krab-dCas9-MeCP2 and CRISPR activation screen (CRISPRa) using dCas9-PP7-p65-VP64 system.

To study the efficiency of HDR in human cells we used the green fluorescent protein (GFP) to blue fluorescent protein (BFP) pair, where conversion from GFP to BFP requires editing of 2 nucleotides. The CRISPR screens were performed on a K562 human cell line stably expressing a green fluorescent protein (GFP). GFP was targeted with Cas9, guide RNA targeting GFP, and a single stranded DNA (ssDNA) encoding BFP. Efficiency of HDR in cells carrying specific genetic perturbations (genetic inhibition or activation) were identified using fluorescence-activated cell sorting (FACS), followed by guide RNA recovery and quantification using next-generation sequencing (NGS).

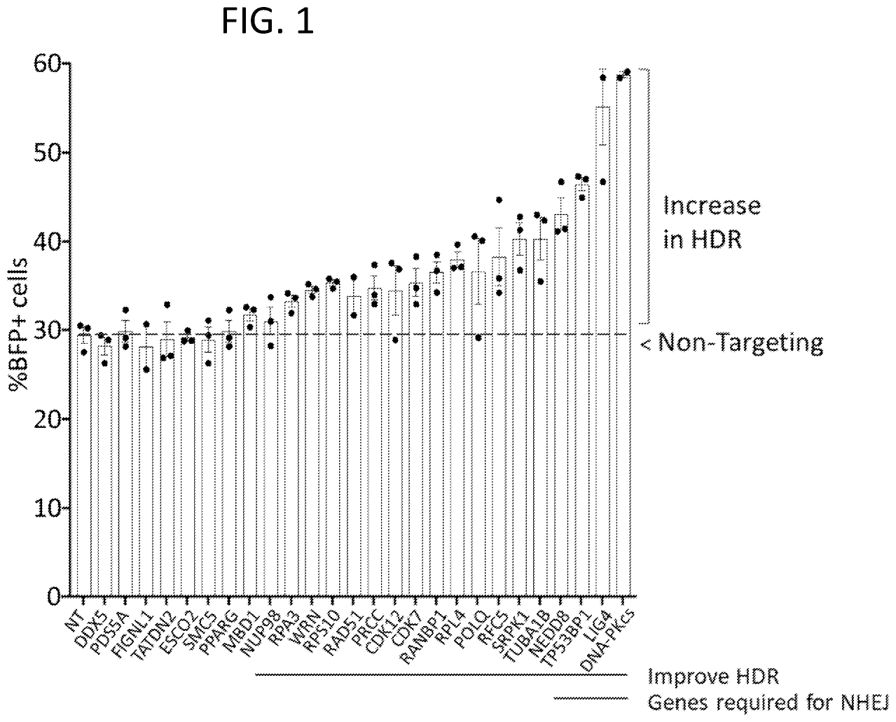

As shown in FIG. 1, the top hits in the CRISPRi arrayed validations were genes involved in Non-Homologous End-Joining (NHEJ). Among the top CRISPRi hits that promote HDR we identified genes involved in DNA damage (DNA-PK, TP53BP1 and LIG4). Knock out of DNA-PK, TP53BP1 and LIG4 showed an increase in HDR levels, as previously established. We identified an additional 13 genes whose knock out promoted HDR in K562 cells. When NHEJ was blocked, the levels of HDR only increased to ˜50-60%. The top 500 genes from the CRISPRi screen which when inhibited increase HDR levels are listed in rank order in the Table 2. The genes are ranked based on log 2-transformed mean guide fold change, where each gene was targeted with 6 individual guides.

The top 500 genes from the CRISPRa screen which, when activated, increase HDR level or efficiency are listed in Table 1. The genes are ranked based on log 2-transformed mean guide fold change, where each gene was targeted with 6 individual guides.

The top CRISPR screen results of the top 200 genes, ranked by fold change, were evaluated through a Reactome analysis. Many of the top genes were involved in specific biological processes such as mRNA splicing, protein translation, cell cycle as shown in the table below:

TABLE 4

| CRISPRi | |
|---|---|
| Screen- Top | |
| 10 Genes | Gene Function |
| MED21 | Mediator complex, RNA transcription |
| LIAS | Lipoic Acid Synthetase |
| DAD1 | Defender against apoptotic cell death |
| GTF2F2 | General Transcription Factor IIF Subunit 2 |
| CSTF3 | Cleavage Stimulation Factor Subunit 3, pre-mRNA |
| | processing |
| RRP36 | Ribosomal RNA Processing 36 |
| RPS10 | Ribosomal Protein S10 |
| SRP14 | Signal Recognition Peptide 14 |
| PRKDC | DNA-PK; DNA-Dependent Protein Kinase Catalytic |
| | Subunit |
| RPS6 | Ribosomal Protein S6 |

### Example 2—DNA-Pk Knock-Out Monoclonal Cell Lines

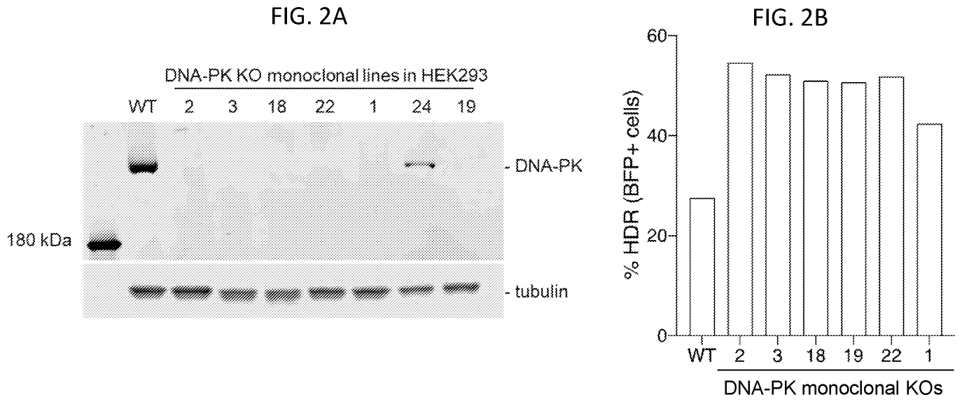

To identify additional regulators of HDR that synergize with NHEJ inhibition and further increase HDR levels in human cells, we generated DNA-PK monoclonal knockout cells. The HEK293 DNA-PK knockout cells were generated by targeting DNA-PK gene in HEK293 cells with a guide and Cas9 nuclease. Monoclonal lines were tested by Western blot to check the expression of DNA-PK at protein level. Wildtype (WT) HEK293 cells show expression of DNA-PK, while the DNA-PK knockout was completely lost in clones 2, 3, 18, 19, and 22 of the Western blot of FIG. 2A. Residual DNA-PK protein levels were detected in clone 1 and 24.

The HDR levels in the DNA-PK monoclonal lines were measured using the green fluorescent protein (GFP)-to-blue fluorescent protein (BFP) conversion assay. Levels of HDR were increased by 2-fold compared to WT cells and consistent across the monoclonal lines with complete loss of DNA-PK expression (clone 2, 3, 18, 19, 22), as shown in the bar graph of FIG. 2B.

### Example 3—Crispr Inhibition Screens in DNA-Pk Ko Cells

In follow-up work, we knocked-out 26 top hit genes from the CRISPRi screen and 13 top hit genes from the CRISPRa screen. Among the genes tested, we validated 16 genes from our loss-of-function (CRISPRi) screen (FIG. 1) and 11 genes from our gain-of-function (CRISPRa) screen. We verified that these genes showed an increase in HDR levels.

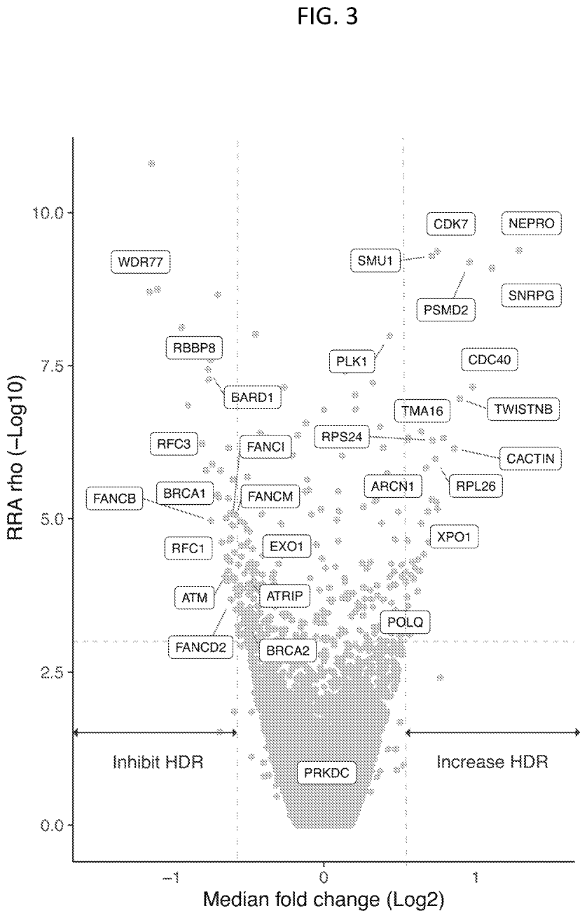

Using DNA-PK knockout clonal lines 18 and 22 as biological replicas, a genome-wide CRISPR inhibition screen was performed in these cells. We identified genes that increase (rightmost third of FIG. 3) and decrease HDR (median fold change). Among the genes that decrease HDR (leftmost third of FIG. 3) are BRCA1, FANCM, FANCI, BARD1, and RBBP8. Among the genes that increase HDR levels are POLQ, XPO1, ARCN1, RPL26, CACTIN, TMA16, RPS24, TWISTNB, CDC40, PSMD2, SNRPG, NEPRO, CDK7 and SMU1.

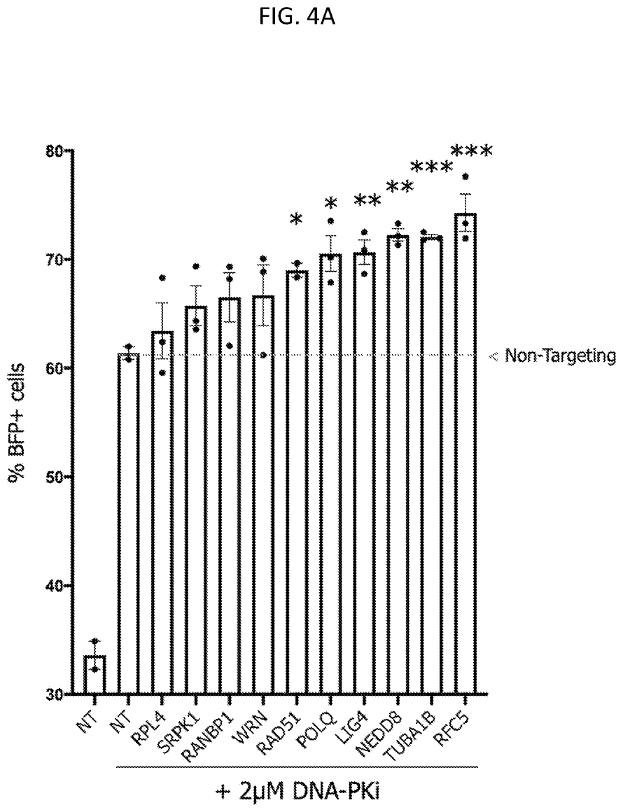

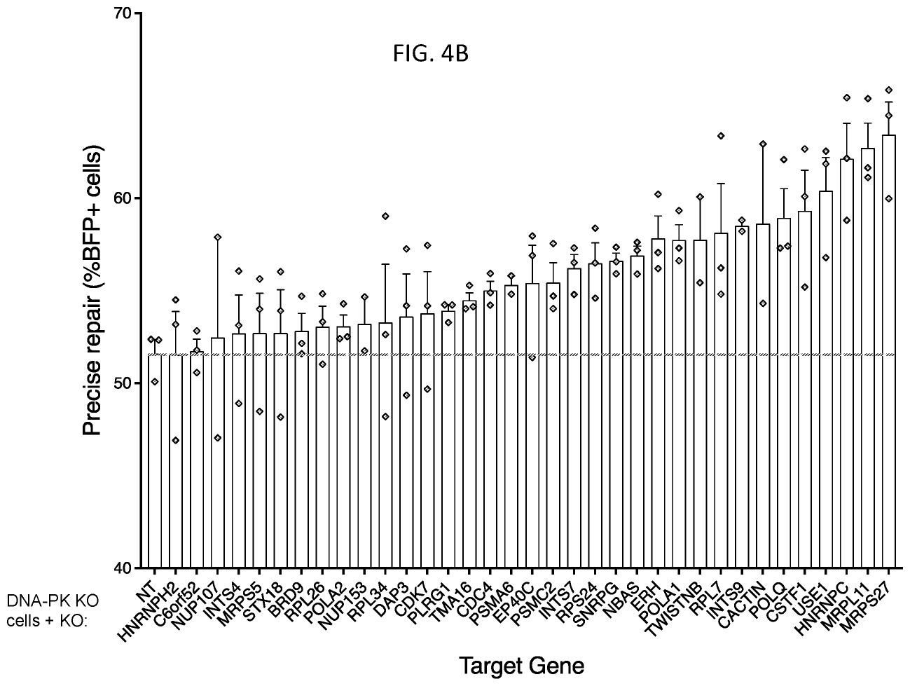

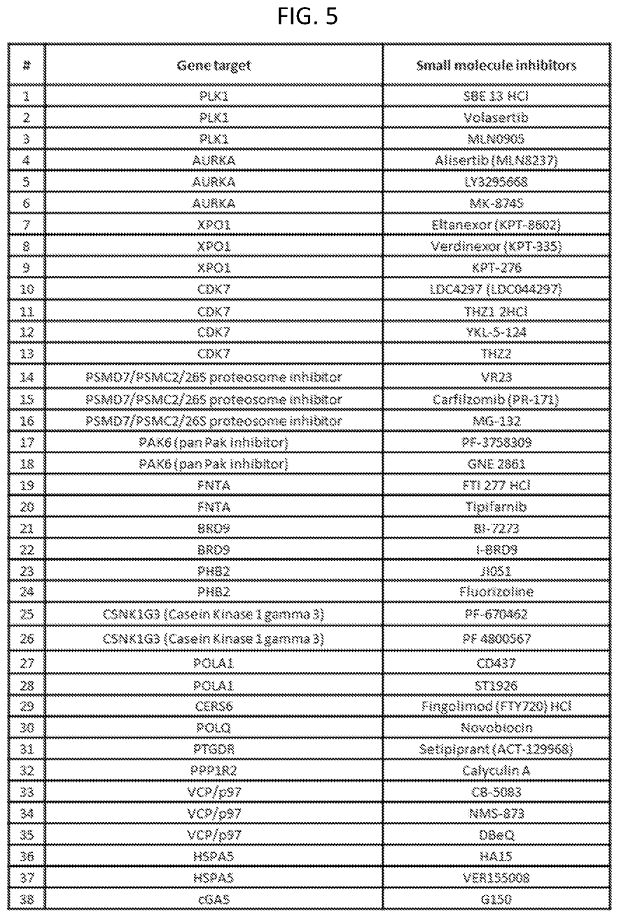

To demonstrate that combinatorial gene perturbation drives significantly higher HDR levels, knock out cell lines of the RPL4, SRPK1, RANBP1, WRN, RAD51, POLZ, LIG4, NEDD8, TUBA1B and RFCS were treated with 2 μM of DNA-PK inhibitor NU7441. HDR levels were determined by cell sorting. As shown in FIG. 4A, blocking DNA-PK in RFC5, TUBA1B, NEDD8, LIG4, POLQ and RAD51 knock-out lines, i.e., a combination of two gene inhibitions/knock outs, resulted in a significant increase in the HDR levels.

Additional results of arrayed validations using the GFP-to-BFP assay identified increases in HDR resulting from inhibition of DNA-PK and a second gene target. Briefly, DNA-PK knockout cells clone 22 were targeted with NT (non-targeting guide as a control) to establish a baseline, or with a guide targeting one of the indicated genes, i.e., MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9, STX18, MRPS5, INTS4, NUP107, C6orf52 or HNRNPH2. As shown in the bar graph of FIG. 4B, most of the genes showed increased HDR levels when perturbed in DNA-PK knockout cells.

### Example 4—Inhibiting Hdr with Combinations of Small Molecule Inhibitors

Known small molecule inhibitors of 38 of the gene targets of Table 2 were purchased from Selleckchem.com, Med ChemExpress and Millipore Sigma for evaluation of the effect of inhibiting two gene targets simultaneously and determining the effect on HDR levels. The list of gene targets and corresponding known small molecule inhibitors are provided in the table of FIG. 5.

The small molecules were tested on DNA-PK knockout HET293 cells at concentrations of 10 μM or 1 μM. Eighteen targets were targeted with 38 drugs. Some drugs were lethal and so were eliminated from use. Drug validations were performed in DNA-PK KO cells clone 22 treated with dimethyl sulfoxide (DMSO) or 1 or 10 μM of the indicated inhibitors. HDR levels were measured with the BFP-to-GFP assay. Small molecule inhibitors were added in the media simultaneously with the introduction of Cas9, guide RNA and single-stranded DNA (ssDNA) encoding BFP. The drugs were washed off 24 hours later.

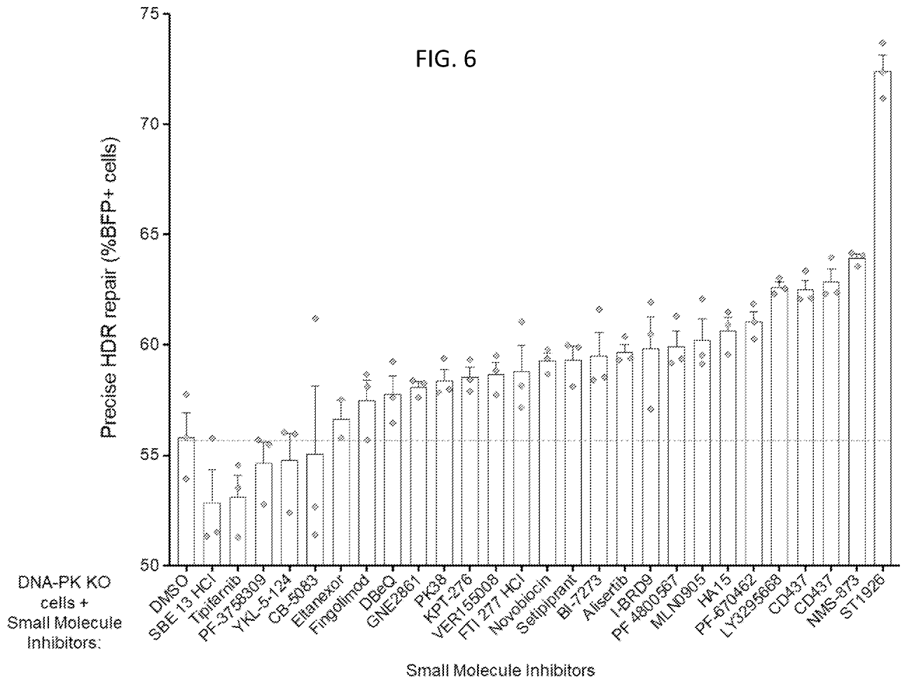

As illustrated in FIG. 6, about 75% of the inhibitor compounds of the indicated gene targets at 1 μM concentrations showed an increased HDR levels. These include
PLK1 inhibitor MLN0905;
AURKA inhibitors Alisertib (MLN8237) and LY3295668;
XPO1 inhibitors Eltanexor (KPT-8602) and KPT-276;
CDK7 inhibitor YKL-5-124;
PAK6 (pan Pak inhibitor) PF-3758309
CERS6 inhibitor Fingolimod (FTY720) HCl
BRD9 inhibitors I-BRD9 and BI-7273
POLA1 inhibitors ST1926 and CD437
VCP/p97 inhibitors NMS-873 and DBeQ;
CSNKIG3 (Casein Kinase 1 gamma 3) inhibitors PF-670462 and PF 4800567
HSPA5 inhibitors HA15 and VER155008
PTGDR inhibitor Setipiprant (ACT-129968)
POLQ inhibitor Novobiocin
FNTA inhibitor FTI 277 HCl and
VCP/p97 inhbitor CB-5083.

These data provide evidence that these combinations of drugs that inhibit at least two gene targets can be useful to enhance HDR efficiency when used in combination with CRISPR gene editing techniques.

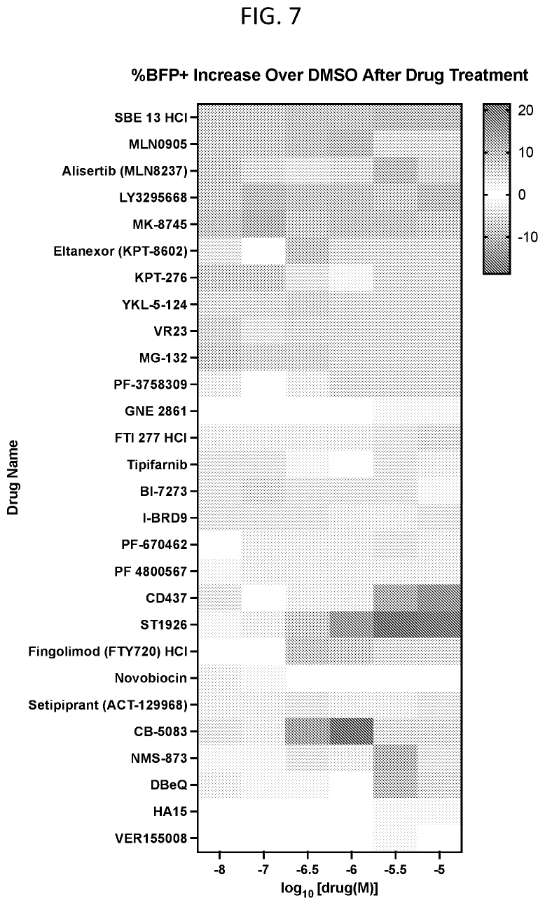

The compounds shown in FIG. 6 were further tested for dose dependent effects. The same cells as described above were treated with the noted compounds at compounds at 10 μM, 5 μM, 1 μM, 0.5 μM, 0.1 μM, and 0.01 μM. HDR levels are shown as a % of BFP+ cells over DMSO 24 hours after drug treatment (FIG. 7).

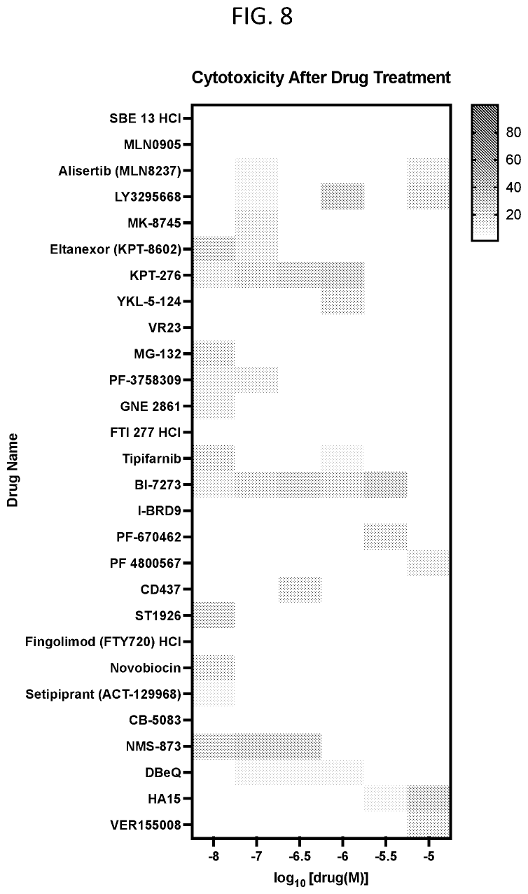

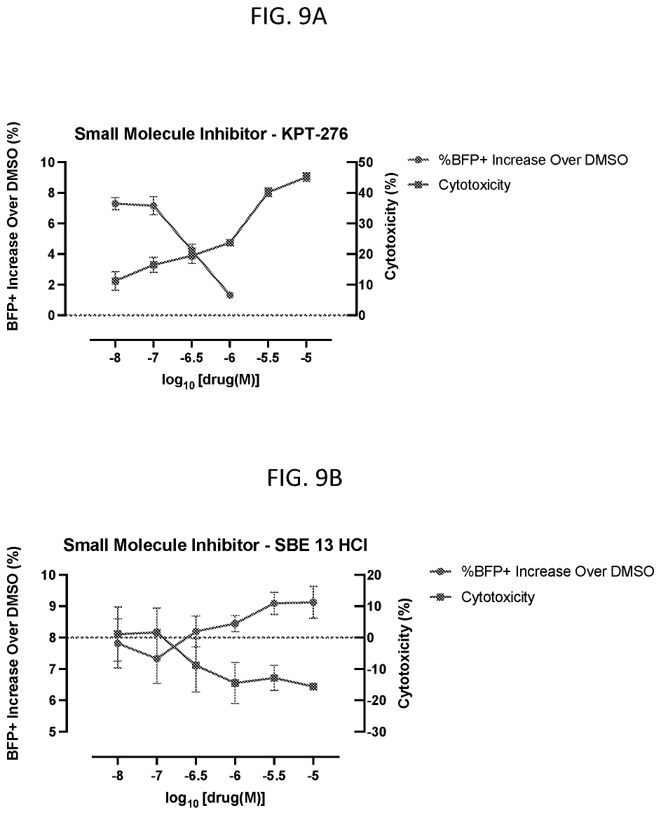

The same compounds were also tested for cytotoxicity at 10 μM, 5 μM, 1 μM, 0.5 μM, 0.1 μM, and 0.01 μM. FIG. 8 shows cytotoxicity after drug treatment. FIG. 9A is a graph showing the BFP+ increase and cytotoxicity over DMSO for compound KPT-276, and FIG. 9B shows results for compound SBE 13 HCl. While some small molecule inhibitors were cytotoxic, especially at high concentrations, it was observed that for compounds that showed a dose-dependent increase in HDR, low toxicity was observed.

### Example 5—Screening of Combinations

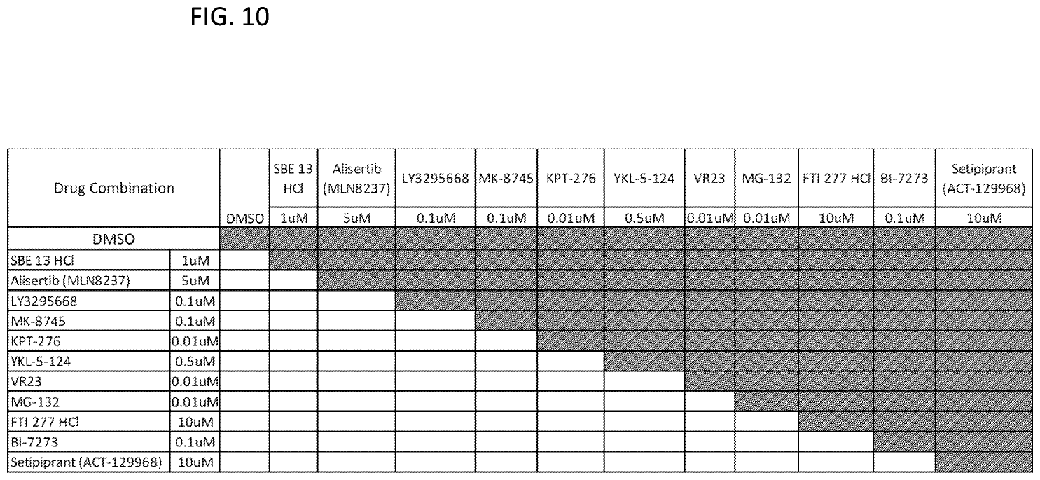

From the results of the above examples, certain combinations are tested to determine the minimum dosage that results in high-level HDR. FIG. 10 shows a table of compound combinations. 11 compounds were selected and are tested in combination at the noted concentrations. The tested compounds and their structures are shown in Table 3.

### Example 6—Gene Editing with Increased Hdr Efficiency

The method of enhancing gene editing efficiency is demonstrated for a human patient suffering from the disease Mucopolysaccharidosis 1 (MPS1). MPS1 arises from mutation in the gene IDUA. IDUA encodes an enzyme called alpha-L-iduronidase that is needed for breakdown of glycosaminoglycans (GAGs). Patients with a mutation in this enzyme, accumulate a large amount of GAG leading to cell, tissue and organ damage. Currently there is no effective treatment for this disease. The life expectancy for children born with this mutation is about 10 years.

To reverse the MPS I disease phenotype, we transiently prime the patient by suppressing or activating gene expression of genes that we identified to regulate homology-directed repair. The priming is done by inhibiting or activating a desired gene. Gene inhibition is done by delivering a small molecule inhibitor, siRNA, antisense oligonucleotide, RNA-targeting enzyme Cas13, or a dCas9 repressor, such as KRAB, MeCP2, etc. Gene activation is accomplished by delivering mRNA, gene ORFs on a plasmid, a dCas9 activator such as p65, HSF1, etc.), or a gene delivery viral based method. The priming methods described here allow for transient and reversable changes allowing for high-efficiency HDR. Once the patient is primed with desired combinations of Inhibitor(s) and/or Activator(s) as described above, a Cas9 enzyme mRNA, guide RNA, and single-stranded DNA template containing the desired DNA edits are delivered via nanoparticle-based methods. Primed patients exhibit high-levels of permanent HDR-based gene editing. The efficiency of gene editing in vivo is tested by tissue biopsy.

### Example 7—Gene Editing with Priming with Combined Inhibitory Compositions for Increased HDR Efficiency

To investigate the effect of combinatorial inhibition using a DNA-PK inhibitor and another inhibitor on HDR gene editing rates, human K562 PRKDC−/− cells (a DNA Knock Out cell line) were nucleofected with SpCas9 RNPs with an EGFP-targeting sgRNA and EBFP ssODN and then incubated with 1 μM (micro molar) concentration ST1926 (an inhibitor of POLA1) or DMSO treated (control). Gene editing rates are expressed in percentages and classified as % precise repair (% BFP conversion). The combination of ST1926 treatment in PRKDC-null human cells boosted precise editing from 56% to 73%. See FIG. 6.

In an analogous manner, a combination of ST1926 and a DNA-PK inhibitor, such as NU7441 are delivered ex vivo to an exogenous T cell obtained from a patient suffering from a cancer for 5 hours at room temperature. The inhibitors are delivered in a pharmaceutically acceptable buffer and excipient. After hour 5, an LNP carrying Cas9mRNA, a gRNA targeting a mutated gene and having a single-stranded DNA template containing the desired DNA edits are delivered via nanoparticle-based methods to the cell ex vivo. Once the target gene is edited, the cells are re-infused into the patient. A protocol similar to this can be used to treat cystic fibrosis, among others, according to current clinical trial protocols. The resulting correction of the mutated gene results in a therapeutic benefit to the patient.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

1. A composition comprising
(a) the components necessary for performing precise gene repair of a target gene; and at least one of
(b) an inhibitory component that temporarily inhibits, down-regulates, or blocks the expression or activity of a gene selected from Table 2;
(c) an activating component that temporarily increases, upregulates or overexpresses the gene product or activity of a gene selected from Table 1: or
(d) a combination of at least one inhibitor component of (b) and at least one activating component of (c),
wherein the presence of (b), (c) or (d) in the composition enables an increase in the efficiency of said precise gene repair of the target gene.

2. The composition according to claim 1, comprising two or more inhibitory components, each component temporarily inhibiting, down-regulating, or blocking the expression or activity of one gene selected from Table 2.

3. The composition according to claim 1 or claim 2, comprising two or more activating components, each component that temporarily increases, upregulates or overexpresses the gene product or activity of one gene selected from Table 1.

4. The composition according to any one of claims 1 to 3, wherein the form of precise gene repair is homology-directed repair (HDR), base editing repair, or prime editing repair.

5. The composition according to any one of claims 1 to 4, wherein the component (a) comprises a CRISPR-associated endonuclease (Cas) protein and a guide RNA sequence that targets said target gene.

6. The composition according to any one of claims 1 to 5, further comprising a delivery vehicle suitable for administration in vivo into a mammalian subject or ex vivo to cells of a mammalian subject.

7. The composition according to claim 6, wherein the delivery vehicle is a polymeric nanoparticle, inorganic nanoparticle, a lipid-based composition, a nanocapsule lipid base, a recombinant viral vector, a recombinant plasmid, a buffer, or a combination thereof.

8. The composition according to claim 1, wherein said inhibitory component comprises an inhibitor of a gene involved in Non-homologous end-joining (NHEJ).

9. The composition according to claim 1, wherein the gene (b) which when inhibited causes an increase in precise editing repair is DNA-PK, LIG4, TP53BP1, NEDD8, TUBA1B, SRPK1, RFC5, POLQ, RPL4, RANBP1, CDK7, CDK12, PRCC, RAD51, RRS10, WRN, RPA3, NUP98, MBD1, PPARG, SMC5, ESCO2, TATDN2, FIGNL1, PDS5A, or DDX5.

10. The composition according to claim 2, wherein the combination of inhibitory components comprises an inhibitor of a gene involved in Non-homologous end-joining (NHEJ) and an inhibitor of one or more additional genes of Table 2, wherein the combination increases the efficiency of said repair.

11. The composition according to claim 10, wherein the additional gene is POLQ, XPO1, RPL26, ARCN1, CACTIN, RPS24, TMA16, TWISTNB, CDC40, PSMD2, SNRPG, SMU1, CDK7 or NEPRO.

12. The composition according to claim 10, wherein the additional gene is MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9, STX18, MRPS5, INTS4, NUP107, C6orf52 or HNRNPH2.

13. The composition according to claim 10, wherein the additional gene of Table 2 is PLK1, AURKA, XPO1, CDK7, PSMC2, FNTA, BRD9 or PTGDR.

14. The composition according to claim 1, comprising an inhibitory component of (b) and an activating component of (c).

15. The composition according to claim 15, wherein the gene of Table 1 is WDR77, RBBP8, RFC3, FANCB, BRCA1, RFC1, ATM, or FANCO2.

16. The composition according to any one of claim 8, or 10 to 13, wherein said gene involved in Non-homologous end-joining (NHEJ) is DNA-PK, LIG4 or TP53BP1.

17. The composition according to any one of claims 1 to 16, wherein the activating or over-expressing component is a small molecule, an mRNA or DNA encoding the additional gene, or the purified protein product of the additional gene.

18. The composition according to any one of claims 1 to 16, wherein the inhibitory components are independently, a small molecule, a nucleic acid, an siRNA, an shRNA, an antisense oligonucleotide, a second RNA guide sequence directed to an additional gene, an antibody, a protein, a plasmid expressing the component as a DNA or protein, or a combination thereof.

19. The composition according to any one of claims 1 to 18, wherein the inhibitory components are one or more of the small molecules of FIG. 4B or 6.

20. A method for increasing the efficiency of precise gene editing of a target gene comprising administering to a mammalian subject in vivo, or contacting mammalian cells ex vivo with,
(a) a composition that temporarily inhibits, down-regulates, blocks or reduces the expression or activity of one gene or a combination of genes selected from Table 2,
(b) a composition that temporarily activates, up-regulates, stimulates or overexpresses the product, expression or activity of at least one additional gene or a combination of additional genes selected from Table 1: or
(c) a composition of (a) and a composition of (b);
said compositions (a), (b), or (c) being administered prior to or simultaneously with the components necessary to perform a gene editing technique and precise editing repair of said target gene.

21. The method according to claim 20, wherein the form of precise gene repair is homology-directed repair (HDR), base editing repair, or prime editing repair.

22. The method according to claim 20 or 21, wherein the gene editing is CRISPR and the components necessary to perform said CRISPR-mediated precise editing repair comprise a CRISPR-associated endonuclease (Cas) protein and a guide RNA sequence.

23. The method according to any one of claim 20 to claim 22, wherein said inhibitory composition (a) or (c) comprises an inhibitor of a gene involved in Non-homologous end-joining (NHEJ).

24. The method according to any one of claims 20 to 23, wherein the gene selected from Table 2 which when inhibited causes an increase in precise editing repair is DNA-PKcs, LIG4, TP53BP1, NEDD8, TUBA1B, SRPK1, RFC5, POLQ, RPL4, RANBP1, CDK7, CDK12, PRCC, RAD51, RRS10, WRN, RPA3, NUP98, MBD1, PPARG, SMC5, ESCO2, TATDN2, FIGNL1, PDS5A, or DDX5.

25. The method according to any one of claims 20 to 24, wherein the composition (a) comprises an inhibitor of a gene involved in Non-homologous end-joining (NHEJ) and an inhibitor of one or more additional genes of Table 2, wherein the combination increases the efficiency of said repair.

26. The method according to any one of claims 20 to 25, wherein the additional gene of Table 2 is POLQ, XPO1, RPL26, ARCN1, CACTIN, RPS24, TMA16, TWISTNB, CDC40, PSMD2, SNRPG, SMU1, CDK7 or NEPRO.

27. The method according to any one of claims 20 to 25, wherein the additional gene of Table 2 is PLK1, AURKA, XPO1, CDK7, PSMC2, FNTA, BRD9 or PTGDR.

28. The method according to any one of claims 20 to 25, wherein the additional gene of Table 2 is MRPS27, MRPL11, HNRNPC, USE1, CSTF1, POLZ, CACTIN, INTS9, RPL7, TWISTNB, POLA1, EFH, NBAS, SNRPG, RPS24, INTS7, PSMC2, EP20C, PSMA6, CDC4, TMA16, PLRG1, CDK7, DAP3, RPL34, NUP153, NUP153, POLA2, RPL26, BRD9, STX18, MRPS5, INTS4, NUP107, C6orf52 or HNRNPH2.

29. The method according to any one of claims 20 to 28, wherein the additional gene of Table 1 is WDR77, RBBP8, RFC3, FANCB, BRCA1, RFC1, ATM, or FANCO2.

29. The method according to any one of claims 20 to 28, wherein the combination of inhibitory and activating components comprises an inhibitor of a gene involved in Non-homologous end-joining (NHEJ) with a component that initiates over-expression of the products of one or more additional genes of Table 1, wherein the combination of the temporary inhibition of the first additional gene and the temporary overexpression of the product of said second additional gene activates the efficiency of the repair.

30. The method according to claim 29, wherein the additional gene of Table 1 is WDR77, RBBP8, RFC3, FANCB, BRCA1, RFC1, ATM, or FANCO2.

31. The method according to claim 23, 25, or 29, wherein said gene involved in Non-homologous end-joining (NHEJ) is DNA-PK, LIG4 or TP53BP1.

32. The method according to any one of claims 20 to 31, wherein the activating or over-expressing component is a small molecule, an mRNA or DNA encoding the additional gene, or the purified protein product of the additional gene that once delivered can increase the cellular level of said additional gene, wherein increased expression of said additional gene or genes activates the efficiency of the repair.

33. The method according to any one of claims 20 to 31, wherein the inhibitory components are independently, a small molecule, a nucleic acid, an siRNA, an shRNA, an anti-sense oligonucleotide, a second RNA guide sequence directed to an additional gene, an antibody, a protein, a plasmid expressing the component as a DNA or protein, or a combination thereof.

34. The method according to any one of claims 20 to 33, wherein the inhibitory compositions comprise one or more of the small molecules of FIG. 4A or 6.

35. The method according to any one of claims 20 to 34, wherein the mammalian cell is an autologous cell obtained from the mammalian subject, subjected to gene editing in vivo, and subsequently transferred in vivo to said mammalian subject.

36. The method according to claim any one of claims 20 to 35, further comprising administering the compositions (a), (b) or (c) between 1 to 24 hours prior to administering the gene editing components.

37. The method according to any one of claims 20 to 36, wherein the gene editing components for repair of the target gene are packaged in a nanoparticle or nanocapsule and delivered to the subject or cells separately from the compositions (a), (b) or (c).

38. The method according to any one of claims 20 to 37, wherein the compositions of (a), (b), or (c) are independently, a small molecule, a nucleic acid sequence, an siRNA, an shRNA, a second RNA guide sequence directed to an additional gene, an antibody, a protein, a plasmid or recombinant virus designed to express the additional gene as a DNA or protein temporarily, or combinations thereof.

39. The method according to any one of claims 20 to 38, wherein said mammalian subject is a human subject that has a genetically mediated disease.

40. The method according to claim 20, wherein said target gene is a gene the mediates or is responsible for a genetically mediated disease in a human subject.

41. The composition or method according to any preceding claim, wherein the inhibitor is SBE 13 HCl, Alisertib, LY3295668, MK-8745, KPT-276, YKL-5-124, VR23, MG132, FTI 277 HCl, BI-7273, or setipiprant.

42. A composition comprising two or more of SBE 13 HCl, Alisertib, LY3295668, MK-8745, KPT-276, YKL-5-124, VR23, MG132, FTI 277 HCl, BI-7273, or setipiprant.
